(19) United States
(12) Patent Application Publication
(10) Pub. No.: US 2024/0423972 A1
(43) Pub. Date: Dec. 26, 2024

(54) CHEMOTHERAPEUTIC BIOADHESIVE PARTICLES WITH IMMUNOSTIMULATORY MOLECULES FOR CANCER TREATMENT

(71) Applicant: Yale University, New Haven, CT (US)

(72) Inventors: Hee-Won Suh, North Haven, CT (US); Julia M. Lewis, Clinton, CT (US); W. Mark Saltzman, New Haven, CT (US); Michael Girardi, Madison, CT (US)

(21) Appl. No.: 18/263,381

(22) PCT Filed: Feb. 1, 2022
(86) PCT No.: PCT/US2022/014676

§ 371 Date: Jul. 28, 2023

Related U.S. Application Data
(60) Provisional application No. 63/144,331, filed on Feb. 1, 2021.

(51) Int. Cl. A61K 31/4745; A61K 9/51; A61K 31/711; A61K 47/54; A61K 47/69; A61P 35/00

(52) U.S. Cl. CPC A61K 31/4745; A61K 9/5153; A61K 31/711; A61K 47/549; A61K 47/6937; A61P 35/00

(57) ABSTRACT

A bioadhesive nanoparticle (BNP) for long-lasting local drug delivery to treat cancer was developed. The bioadhesive nanoparticles (BNP) are composed of biodegradable polymer such as poly(lactic acid)-hyperbranched polyglycerol (PLA-HPG), encapsulating a chemotherapeutic such as camptothecin (CPT). Nanoparticles (NPs) of PLA-HPG are non-adhesive NPs (NNPs), which are stealthy in their native state, but conversion of the vicinal diols of HPG to aldehydes confers the ability to form strong covalent bonds with amine-rich surfaces. The formulation is administered in combination with immunostimulatory molecules such as CPG and shows unexpectedly better killing of cancer cells.

### CROSS-REFERENCE TO RELATED APPLICATION

This application claims the benefit of and priority to U.S. Ser. No. 63/144,331 filed Feb. 1, 2021, and which is incorporated by referenced herein in its entirety.

### FIELD OF THE INVENTION

The present invention is in the field of chemotherapy, and more particularly in the area of enhanced treatment of cancers, especially skin cancers.

### STATEMENT REGARDING FEDERALLY FUNDED RESEARCH

None.

### BACKGROUND OF THE INVENTION

Keratinocyte-derived carcinomas, including squamous cell carcinoma (SCC), are among the most common malignancies. Surgical excision is the therapeutic standard but is not always clinically feasible, and currently available alternatives are limited to superficial tumors. Even with chemotherapy and/or radiation, outcomes are not always favorable.

In the United States, the frequency of skin malignancies exceeds the frequency of all other cancers combined. Keratinocyte-derived carcinomas (KDCs), such as cutaneous squamous cell carcinoma (SCC), comprise the most common class of these malignancies, with SCCs responsible for approximately 5 million cases and 10,000 deaths annually in the United States. The incidence of cutaneous SCC has been steadily rising, with some estimates reporting greater than a 250% increase from 1976 to 1984 and 2000 to 2010. These numbers are projected to rise in proportion to the expanding elderly population, as well as the number of immunocompromised patients living with advanced disease, posing a significant public health challenge. Surgical excision is the most common first-line treatment for cutaneous SCC; however, SCC recurrence is common, with one study reporting rates as high as 50% of patients. Furthermore, surgical excision is undesirable in certain clinical settings, including at-risk patients with underlying bleeding diatheses and large-diameter tumors requiring complicated wound closures that increase the potential for postsurgical complications. The management and treatment of KDCs accounts for over $8.1 billion in US healthcare expenditures annually, a significant burden on both individuals and healthcare systems, necessitating efficient and effective alternatives.

Although both topical chemotherapeutic and immunomodulatory agents have demonstrated potential in the local treatment of superficial SCCs (Salim, Br. J. Dermatol. 148, 539-543 (2003); Morton Arch. Dermatol. 142, 729-735 (2006); Patel J. Am. Acad. Dermatol. 54, 1025-1032 (2006); Mackenzie-Wood, J. Am. Acad. Dermatol. 44, 462-470 (2001)), cream and gel formulations fail to achieve adequate penetration into deeper (e.g., nodular) SCCs. Moreover, topical chemotherapy (e.g., 5-fluorouracil) and immunotherapy (e.g., imiquimod) can diffuse from the site of application into the dense vasculature within tumors, with potential for diminished local efficacy and systemic toxicity (Kishi, Drug Saf. Case Rep. 5, 4 (2018). Thus, novel strategies for local drug delivery are warranted.

Nanoparticles (NPs) formed from the block copolymer poly(lactic acid)-hyperbranched polyglycerol (PLA-HPG) have been shown to increase the duration, bioavailability, and efficacy of locally administered chemotherapy drugs, which minimizes the systemic toxicity associated with conventional chemotherapies. PLA-HPG NPs are produced as nonadhesive nanoparticles (NNPs) that can then be transformed into bioadhesive nanoparticles (BNPs) by a brief incubation with sodium periodate (Deng et al., Proc. Natl. Acad. Sci. U.S.A. 113, 11453-11458 (2016). (FIG. 1A). This treatment alters the chemistry of the surface HPG molecules, converting the vicinal diols to aldehydes, which are capable of forming strong, covalent bonds with amines on the tumor cell surface and tumor interstitial matrix proteins. This bioadhesive effect leads to durable association of BNPs with cells, stratum corneum, mesenteric membranes, vaginal epithelium, and intracranial tumor cells. BNPs, applied as a topical sunscreen, can prevent the doublestranded DNA breaks associated with cutaneous carcinogenesis, while at the same time minimizing the systemic accumulation of ultraviolet filters common to commercially available sunscreens (Deng et al., Nat. Mater. 14, 1278-1285 (2015); Suh et al., Bioeng. Transl. Med. 4, 129-140 (2018)).

### SUMMARY OF THE INVENTION

A bioadhesive nanoparticle (BNP) for long-lasting local drug delivery to treat cancer was developed. The bioadhesive nanoparticles (BNP) are composed of biodegradable polymer such as poly(lactic acid)-hyperbranched polyglycerol (PLA-HPG), encapsulating a chemotherapeutic such as camptothecin (CPT). Nanoparticles (NPs) of PLA-HPG are non-adhesive NPs (NNPs), which are stealthy in their native state, but conversion of the vicinal diols of HPG to aldehydes confers the ability to form strong covalent bonds with amine-rich surfaces.

The formulation is administered in combination with immunostimulatory molecules such as CPG and shows unexpectedly better killing of cancer cells. This was demonstrated by incorporating the topoisomerase inhibitor camptothecin (CPT) into bioadhesive nanoparticles (“BNPs”) formed of hyperbranched polyglycerols (“HPG”) having some to all of the hydroxyl groups functionaled to be bioadhesive. These BNPs show enhanced tumor cell uptake, bioadhesion within the tumor microenvironment, and prolonged intratumoral drug retention.

These BNPs have significantly enhanced binding to Keratinocyte-derived carcinomas (KDCs), such as cutaneous squamous cell carcinoma (SCC). and SCC tumor cell surfaces and matrix proteins, thereby significantly enhancing the therapeutic efficacy of intratumoral drug delivery. Tumor injection of BNP-CPT resulted in tumor retention of CPT at ˜50% at 10 days postinjection, while CPT was undetectable in NNP-CPT or free (intralipid) CPT-injected tumors at that time. BNP-CPT also significantly reduced tumor burden, with a portion (˜20%) of BNPCPT-treated established tumors showing histologic cure. Larger, more fully established PDV SCC tumors treated with a combination of BNP-CPT and immunostimulating CpG oligodeoxynucleotides exhibited enhanced survival relative to controls, demonstrating the benefit for BNP delivery to be used along with local tumor immunotherapy.

The examples show that BNPs encapsulating CPT were very effective as a local, nonsurgical treatment for nodular squamous cell carcinoma (SCC) skin cancers in a mouse model. BNP-CPT treatment facilitated tumor destruction and resolution, and was compatible with local immunotherapy. These findings demonstrate that BNP delivery of antitumor agents may provide opportunities for nonsurgical treatment of nodular skin cancers like SCC. Results show that efficacy was more than additive when the BNP delivering chemotherapeutic agent were co-administered with immunostimulatory agents such as CPG.

Taken together, these results show that percutaneous delivery of a chemotherapeutic agent via BNPs, with or without an immunomodulator and/or adjuvant immunostimulation, represents a viable, nonsurgical alternative for treating cutaneous malignancy.

### BRIEF DESCRIPTION OF THE DRAWINGS

FIGS. 1A-1E. Synthesis and bioadhesion of CPT-loaded PLA-HPG NNPs and BNPs. (1A) NP synthesis schematic. (1B) Transmission electron microscopy images of NNP-CPT and BNP-CPT particles. Following NNP oxidation with sodium periodate, BNPs retain their spherical morphology, indicating that conversion does not compromise the structure of the BNPs. (Scale bar: 100 nm.) (1C) CPT-loaded NNPs and BNPs show similar temporal drug release kinetics in 1×PBS at 37° C. Both NNP-CPT and BNP-CPT show the gradual release of drug over 24 h. (1D) Quantification of bioadhesion of NNPs and BNPs in proteinaceous environments following rigorous washing. When quantifying bioadhesion, (1E) BNP-CPT demonstrated superior adherence compared to NNP-CPT. Data normalized to unwashed controls. ****P<0.0001, Student's t test; n=5.

FIGS. 2A-2D. BNPs readily associate with tumor cells, shown by graphs comparing NP association with tumor cells following short (up to 5 h) incubation in serum-containing vs. serum-free conditions (2A, 2B). Baseline fluorescence of both dye-conjugated (Cy5) and -loaded (DiD) NNP and BNP before incubation is similar. Cellular association of Cy5-NPs (Left) and DiD-NPs (Right) with extended incubation in serum containing media shows that even with prolonged exposure, BNPs associate with tumor cells more strongly than NNPs (2C, 2D). MFI, mean fluorescence intensity; n=3 replicates. ***P<0.001, Student's t test.

FIGS. 3A-3E. BNPs facilitate both active internalization by tumor cells and binding to extracellular proteins to promote cytotoxicity. (3A) Effects of temperature on NNP-Cy5 and (3B) BNP-Cy5 association with tumor cells. PDV cells cultured with either NNP-Cy5 or BNP-Cy5 at 4° C. in serum-containing media show only minimal cellular association, whereas 37° C. cultures show significant NP association over time, indicating active cellular processes are required. MFI, mean fluorescence intensity; n=3. (3C) Interrogation of clathrin- or macropinocytosis-mediated mechanisms of cellular uptake. Cells were cultured in serum containing media with NNP-Cy5 or BNP-Cy5 for 16 h and during the final 6 h of culture, vehicle (DMSO, 0.1%), CPZ (20 μM), or EIPA (50 μM) added. Results are expressed as the percent inhibition of NP uptake relative to vehicle control; n=3. (3D) Poly-L-lysine-coated plates were incubated with blank (unloaded) NP, free CPT, NNP-CPT, or BNP-CPT for 1 h. These solutions were then aspirated and PDVC57 seeded in fresh media and cultured for 48 h, when viability was assessed. (3E) PDVC57 was incubated with free CPT, NNP-CPT, or BNP-CPT for 1 h. The CPT solutions were then aspirated and replaced with fresh media for 48 h, when viability was assessed. BNP-CPT increased tumor cell cytotoxicity compared to all other groups; n=3. *P<0.05, **P<0.01, and ****P<0.0001, Student's t test.

FIGS. 4A-4C. BNPs exhibit greater association with tumor microenvironments in vivo to maintain local drug concentrations. Confocal microscopy of PDVC57 tumors and overlying skin 72 h after injection of NNP-Cy5 (4A) or BNP-Cy5 (4B). BNP-Cy5 exhibited greater intratumoral retention when compared to NNP-Cy5 (fluorescence integrated density/tissue area: NNP, 3,541.88; BNP, 10,402.26). (Scale bar: 200 μm.) (4C) CPT recovered from tumors after treatment with intralipid-CPT (IL-CPT), NNP-CPT, or BNP-CPT. At 48 and 240 h following treatment, a greater percentage of the injected CPT was recovered from tumors treated with BNP-CPT (48 h, 72.4%; 240 h, 44.7%) when compared to free CPT (48 h, nd [not detected]; 240 h, nd) and NNP-CPT (48 h, 13.4%; 240 h, nd), indicating enhanced BNP-CPT association within SCC tumors. **P<0.01, ****P<0.0001, Student's t test; n=5

FIGS. 5A-5D. Treatment of murine SCC with intratumoral BNP-CPTs is superior to NNP-CPT or intralipid-CPT. (5A) Experiment timeline. Tumors injected with BNP-CPT showed significantly delayed tumor growth (5B) and reduced tumor weight at harvest (5C); when compared to groups treated with either blank BNPs, IL-CPT, or NNP-CPT. Combination therapy with BNP-CPT (12.5 mg/kg CPT) plus the TLR9 agonist, CpG (10 μg), significantly delayed tumor growth (5D). *P<0.05, **P<0.01, Student's t test (C), Mantel-Cox test (B and D; n=10).

FIGS. 6A-6B. Mice treated with BNP-CPT plus CpG show enhanced survival when compared to BNP-CPT alone (6B), with full resolution of a proportion of tumors. 6-7 mm tumors treated with BNP-CPT (12.5 mg/kg, 4.8% CPT loading) and adjuvant CpG (10 μg) demonstrated improved survival when compared to tumors treated with vehicle, or BNP-CPT alone. Resolution was assessed clinically (6B), as well as histologically (6A), and observed only within mice treated with combination therapy of BNP-CPT and CpG. PBS vs BNP-CPT+CpG *P<0.05, PBS vs BNP-CPT **P<0.01, Mantel-Cox test, n=7 (PBS), 8 (BNP-CPT), 6 (BNP-CPT+CpG).

FIGS. 7A-7D are graphs showing the effects of serum on NP association with tumor cells at early timepoints. PDV was cultured with NNP-Cy5 or BNP-Cy5 (1 mg/ml NP) for 2 or 5 hr in CRPMI containing 10% FBS (7A, 7C) or prepared without serum (7B, 7D) and NP association with cells analyzed by flow cytometry (7A, 7B). ΔMFI=test−No NP control, n=3, **P<0.01, ***P<0.001. Baseline fluorescence of the Cy5-NPs (1 mg/ml) is similar (7C, 7D).

FIGS. 8A-8D are schematic of complementary in vitro cytotoxicity. FIG. 8A shows a simulated, protein-rich tumor matrix created by coating a 96 well-plate with 0.01% poly-l-lysine in 1×PBS. Blank NNPs, blank BNPs, free CPT, NNP-CPT, or BNP-CPT (5 μM CPT) were added to the wells for an hour-long incubation, and then carefully aspirated. Following aspiration, PDVC57 cells were plated on the pre-treated wells (5000 cells/well) in CRPMI, and incubated for 48 hours. After 48 hours, cell viability was assessed using Cell-Titer Glo. (8B) PDVC7 cells were harvested and seeded on a 96-well plate (5000 cells/well). Free CPT, NNP-CPT, or BNP-CPT (5 μM CPT) were added to the wells for an hour-long incubation, then carefully aspirated and the media replaced. After 48 hours, cell viability was assessed using Cell-Titer Glo. FIG. 8B is a control for the first method (8A) in which there is no poly-l-lysine attached to the plate. FIG. 8C is a control showing the poly-l-lysine coated plates incubated with blank (unloaded) NP, free CPT, NNP-CPT or BNP-CPT, but these solutions are not aspirated before the addition of PDVC57 cells. FIG. 8D is a graph of the viability results, showing the control assay demonstrates comparable killing of PDVC57 by free CPT, NNP-CPT and BNP-CPT, and that there is no toxicity associated with unloaded NNP-blank or BNP-blank particles.

### DETAILED DESCRIPTION OF THE INVENTION

### I. Definitions

The term “biocompatible” as used herein refers to one or more materials that are neither themselves toxic to the host (e.g., an animal or human), nor degrade (if the material degrades) at a rate that produces monomeric or oligomeric subunits or other byproducts at toxic concentrations in the host.

The term “biodegradable” as used herein means that the materials degrade or break down into its component subunits, or digestion, e.g., by a biochemical process, of the material into smaller (e.g., non-polymeric) subunits.

The terms “bioactive agent” and “active agent”, as used interchangeably herein, include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they localize in a predetermined physiological environment.

As used herein, “controlled release” refers to a release profile of an agent for which the agent release characteristics of time course and/or location are chosen to accomplish therapeutic or convenience objectives not offered by conventional topical formulations.

“Sustained release” as used herein refers to release of a substance over an extended period of time in contrast to a bolus type administration in which the entire amount of the substance is made biologically available at one time.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

The term “small molecule”, as used herein, generally refers to an organic molecule that is less than about 2000 g/mol in molecular weight, less than about 1500 g/mol, less than about 1000 g/mol, less than about 800 g/mol, or less than about 500 g/mol. Small molecules are non-polymeric and/or non-oligomeric.

The term “copolymer” as used herein, generally refers to a single polymeric material that is comprised of two or more different monomers. The copolymer can be of any form, such as random, block, graft, etc. The copolymers can have any end-group, including capped or acid end groups.

“Hydrophilic,” as used herein, refers to the property of having affinity for water. For example, hydrophilic polymers (or hydrophilic polymer segments) are polymers (or polymer segments) that are primarily soluble in aqueous solutions and/or have a tendency to absorb water. In general, the more hydrophilic a polymer is, the more that polymer tends to dissolve in, mix with, or be wetted by water.

“Hydrophobic,” as used herein, refers to the property of lacking affinity for, or even repelling water. For example, the more hydrophobic a polymer (or polymer segment), the more that polymer (or polymer segment) tends to not dissolve in, not mix with, or not be wetted by water.

Hydrophilicity and hydrophobicity can be spoken of in relative terms, such as, but not limited to, a spectrum of hydrophilicity/hydrophobicity within a group of polymers or polymer segments. In some embodiments wherein two or more polymers are being discussed, the term “hydrophobic polymer” can be defined based on the polymer's relative hydrophobicity when compared to another, more hydrophilic polymer.

The term “lipophilic”, as used herein, refers to compounds having an affinity for lipids.

The term “amphiphilic”, as used herein, refers to a molecule combining hydrophilic and lipophilic (hydrophobic) properties.

The term “microspheres” is art-recognized, and includes substantially spherical colloidal structures formed from biocompatible polymers having a size ranging from about one or greater up to about 1000 microns. In general, “microcapsules,” also an art-recognized term, may be distinguished from microspheres, as formed of a core and shell. The term “microparticles” is also art-recognized, and includes microspheres and microcapsules, as well as structures that may not be readily placed into either of the above two categories, all with dimensions on average of less than about 1000 microns. If the structures are less than about one micron in diameter, then the corresponding art-recognized terms “nanosphere,” “nanocapsule,” and “nanoparticle” may be utilized. In certain embodiments, the nanospheres, nanocapsules and nanoparticles have an average diameter of about 500 nm, 200 nm, 100 nm, 50 nm, 10 nm, or 1 nm.

A composition containing microparticles or nanoparticles may include particles of a range of particle sizes. In certain embodiments, the particle size distribution may be uniform, e.g., within less than about a 20% standard deviation of the mean volume diameter, and in other embodiments, still more uniform, e.g., within about 10% of the median volume diameter.

The term “particle” as used herein refers to any particle formed of, having attached thereon or thereto, or incorporating a therapeutic, diagnostic or prophylactic agent.

“Mean particle size” as used herein, generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

“Monodisperse” and “homogeneous size distribution”, are used interchangeably herein and describe a population of nanoparticles or microparticles where all of the particles are the same or nearly the same size. As used herein, a monodisperse distribution refers to particle distributions in which 90% or more of the distribution lies within 15% of the median particle size, more preferably within 10% of the median particle size, most preferably within 5% of the median particle size.

“Branch point”, as used herein, refers to a portion of a polymer-drug conjugate that serves to connect one or more hydrophilic polymer segments to one or more hydrophobic polymer segments.

The term “targeting moiety” as used herein refers to a moiety that localizes to or away from a specific locale. The moiety may be, for example, a protein, nucleic acid, nucleic acid analog, carbohydrate, or small molecule. Said entity may be, for example, a therapeutic compound such as a small molecule, or a diagnostic entity such as a detectable label. The locale may be a tissue, a particular cell type, or a subcellular compartment. In one embodiment, the targeting moiety directs the localization of an active entity. The active entity may be a small molecule, protein, polymer, or metal. The active entity may be useful for therapeutic, prophylactic, or diagnostic purposes.

The term “reactive coupling group”, as used herein, refers to any chemical functional group capable of reacting with a second functional group to form a covalent bond. The selection of reactive coupling groups is within the ability of the skilled artisan. Examples of reactive coupling groups can include primary amines (—NH₂) and amine-reactive linking groups such as isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters. Most of these conjugate to amines by either acylation or alkylation. Examples of reactive coupling groups can include aldehydes (—COH) and aldehyde reactive linking groups such as hydrazides, alkoxyamines, and primary amines. Examples of reactive coupling groups can include thiol groups (—SH) and sulfhydryl reactive groups such as maleimides, haloacetyls, and pyridyl disulfides. Examples of reactive coupling groups can include photoreactive coupling groups such as aryl azides or diazirines. The coupling reaction may include the use of a catalyst, heat, pH buffers, light, or a combination thereof.

The term “protective group”, as used herein, refers to a functional group that can be added to and/or substituted for another desired functional group to protect the desired functional group from certain reaction conditions and selectively removed and/or replaced to deprotect or expose the desired functional group. Protective groups are known to the skilled artisan. Suitable protective groups may include those described in Greene, T. W. and Wuts, P. G. M., Protective Groups in Organic Synthesis, (1991). Acid sensitive protective groups include dimethoxytrityl (DMT), tert-butylcarbamate (tBoc) and trifluoroacetyl (tFA). Base sensitive protective groups include 9-fluorenylmethoxycarbonyl (Fmoc), isobutyrl (iBu), benzoyl (Bz) and phenoxyacetyl (pac). Other protective groups include acetamidomethyl, acetyl, tert-amyloxycarbonyl, benzyl, benzyloxycarbonyl, 2-(4-biphEnylyl)-2-propyloxycarbonyl, 2-bromobenzyloxycarbonyl, tert-butyl tert-butyloxycarbonyl, l-carbobenzoxamido-2,2.2-trifluoroethyl, 2,6-dichlorobenzyl, 2-(3,5-dimethoxyphenyl)-2-propyloxycarbonyl, 2,4-dinitrophenyl, dithiasuccinyl, formyl, 4-methoxybenzenesulfonyl, 4-methoxybenzyl, 4-methylbenzyl, o-nitrophenylsulfenyl, 2-phenyl-2-propyloxycarbonyl, α-2,4,5-tetramethylbenzyloxycarbonyl, p-toluenesulfonyl, xanthenyl, benzyl ester, N-hydroxysuccinimide ester, p-nitrobenzyl ester, p-nitrophenyl ester, phenyl ester, p-nitrocarbonate, p-nitrobenzylcarbonate, trimethylsilyl and pentachlorophenyl ester.

“Stealth”, as used herein, refers to the property of nanoparticles. These nanoparticles are not cleared by the mononuclear phagocyte system (MPS) due to the presence of the hydroxyl groups. The stealth particles resist non-specific protein absorption.

“About” is intended to describe values either above or below the stated value in a range of approx. +/−10%. The ranges are intended to be made clear by context, and no further limitation is implied. The use of any and all examples, or exemplary language (e.g., “such as”) provided herein, is intended merely to better illuminate the description and does not pose a limitation on the scope of the description unless otherwise claimed.

The phrase “pharmaceutically acceptable” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “pharmaceutically acceptable salts” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts.

The term “treating” preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain.

The term “therapeutically effective amount” refers to an amount of the therapeutic agent that, when incorporated into and/or onto particles described herein, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more diseases or disorders of the brain, such as reducing tumor size (e.g., tumor volume) or reducing or diminishing one or more symptoms of a neurological disorder, such as memory or learning deficit, tremors or shakes, etc. In still other embodiments, an “effective amount” refers to the amount of a therapeutic agent necessary to repair damaged neurons and/or induce regeneration of neurons.

The terms “incorporated” and “encapsulated” refers to incorporating, formulating, or otherwise including an active agent into and/or onto a composition that allows for release, such as sustained release, of such agent in the desired application. The terms contemplate any manner by which a therapeutic agent or other material is incorporated into a polymer matrix, including for example: attached to a monomer of such polymer (by covalent, ionic, or other binding interaction), physical admixture, enveloping the agent in a coating layer of polymer, and having such monomer be part of the polymerization to give a polymeric formulation, distributed throughout the polymeric matrix, appended to the surface of the polymeric matrix (by covalent or other binding interactions), encapsulated inside the polymeric matrix, etc. The term “co-incorporation” or “co-encapsulation” refers to-the incorporation of a therapeutic agent or other material and at least one other therapeutic agent or other material in a subject composition.

More specifically, the physical form in which any therapeutic agent or other material is encapsulated in polymers may vary with the particular embodiment. For example, a therapeutic agent or other material may be first encapsulated in a microsphere and then combined with the polymer in such a way that at least a portion of the microsphere structure is maintained. Alternatively, a therapeutic agent or other material may be sufficiently immiscible in the polymer that it is dispersed as small droplets, rather than being dissolved, in the polymer.

### II. Compositions

### A. Bioadhesive HPG Nanoparticles

Core-shell particles, such as microparticles and nanoparticles, and methods of making and using thereof are described herein. The core is formed of or contains a hydrophobic material, preferably polymeric. The shell is formed of or contains a hyperbranched polymer (HP) with hydroxyl groups, such as a hyperbranched polyglycerol (HPG), hyperbranched peptides (HPP), hyperbranched oligonucleotides (HON), hyperbranched polysaccharides (HPS), and hyperbranched polyunsaturated or saturated fatty acids (HPF). The HP can be covalently bound to the one or more materials that form the core such that the hydrophilic HP is oriented towards the outside of the particles and the hydrophobic material oriented to form the core.

The HP coating can be modified to adjust the properties of the particles. For example, unmodified HP coatings impart stealth properties to the particles which resist non-specific protein absorption and are referred to as non-bioadhesive nanoparticles (NNPs). Alternatively, the hydroxyl groups on the HP coating can be chemically modified to form functional groups that react with functional groups on tissue or otherwise interact with tissue to adhere the particles to the tissue, cells, or extracellular materials, such as proteins. Such functional groups include, but are not limited to, aldehydes, amines, and O-substituted oximes. Particles with an HP coating chemically modified to form functional groups are referred to as bioadhesive nanoparticles (BNPs). The chemically modified HP coating of BNPs forms a bioadhesive corona of the particle surrounding the hydrophobic material forming the core.

Topical formulations containing these HP coated nanoparticles are useful for application to the skin of encapsulated therapeutic, prophylactic, cosmeceutical, or diagnostic agents. Nanoparticles with unusually strong bioadhesive properties do not diffuse into hair follicles and are useful as sunscreens, insect repellant, in cosmetic applications or for the delivery of cosmetic, therapeutic, prophylactic, diagnostic or nutraceutical agents.

The hydrophobic core of the NNPs and BNPs may include one or more active agents, preferably the chemotherapeutica agents. The core may be varied in size. The core may be formed of two or more layers of hydrophobic material containing the agent, so that the release of the active agent is controlled. For example, the size of the core may be varied to vary the depth of penetration of the NNPs into the follicles following topical administration of a formulation containing the NNPs. The core may be formed for extended release of the active agent, so that the active agent is not released, or released within a defined time period such as 2, 4, 8, or 24 hours following application of the topical formulation containing the NNPs and/or BNPs. In other embodiments, the core may be formed of two or more layers of hydrophobic material, each layer containing one or more different agents, and each layer releasing the one or more different agents at specific times for controlled release.

In a preferred embodiment, the BNP are formulated with and/or have bound to their surface immunostimulatory molecules.

Typically, the NNPs are non-adherent to biological tissues and display follicular penetrance, while BNPs are adherent to biological tissues and display no or minimal follicular penetrance. The HP coating can be partially chemically modified to provide a varying degree of adherence of the particles to biological tissues, cells, or extracellular materials, such as proteins.

The BNP may also contain diagnostic agent. Representative classes of diagnostic materials include paramagnetic molecules, fluorescent compounds, magnetic molecules, and radionuclides. Exemplary materials include, but are not limited to, metal oxides, such as iron oxide, metallic particles, such as gold particles, etc. Biomarkers can also be conjugated to the surface for diagnostic applications.

### B. Chemotherapeutic Agents

Agents to be delivered include proteins, peptides, nucleic acid molecules, and organic molecules, as well as diagnostic agents. Anti-cancer drugs (referred to herein as “chemotherapeutics”), include cytotoxic drugs such as doxorubicin, cyclosporines, mitomycin C, cisplatins and carboplatins, BCNU (Bis-chlorethylnitrosourea, carmustine A chemotherapeutic related to lomustine-CCNU and semustine, which partially overlaps the activity/toxicity of alkylating agents, primarily used to treat Hodgkin's disease, melanoma, myeloma, brain tumors), 5FU (5-Fluorouracil is also known as FU or 5FU and is one of the most commonly used drugs to treat cancers including breast cancer and head and neck cancers), methotrexate, adriamycin, camptothecins, epothilones A-F, and taxol), antibodies and bioactive fragments thereof (including humanized, single chain, and chimeric antibodies), and oligonucleotide drugs (including DNA, RNAs including mRNAs, antisense, siRNA, miRNA, anti-miRNA, piRNA, aptamers, ribozymes, external guide sequences for ribonuclease P, and triplex forming agents such as tcPNAs). In some embodiments, the active agent is a vector, plasmid, or other polynucleotide encoding an oligonucleotide such as those discussed above.

Representative anti-cancer agents include, but are not limited to, alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel, epothilones A-F, and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), maytansines, auristatins, and combinations thereof.

Other suitable anti-cancer agents include angiogenesis inhibitors including antibodies to vascular endothelial growth factor (VEGF) such as bevacizumab (AVASTIN®), other anti-VEGF compounds; thalidomide (THALOMID®) and derivatives thereof such as lenalidomide (REVLIMID®); endostatin; angiostatin; receptor tyrosine kinase (RTK) inhibitors such as sunitinib (SUTENT®); tyrosine kinase inhibitors such as sorafenib (NEXAVAR®), erlotinib (TARCEVA®), pazopanib, axitinib, and lapatinib; and transforming growth factor-α or transforming growth factor-O inhibitors.

Camptothecin (CPT) is a DNA topoisomerase I inhibitor and potent chemotherapy whose functionality is limited by its hydrophobicity and poor solubility, rapid physiological inactivation, and high risk for systemic toxicity. The examples demonstrate that encapsulation of CPT within BNPs (BNP-CPT) enhances the delivery of CPT, resulting in greater tumor cell uptake, tumor drug retention, and tumor elimination relative to free (intralipid) drug or NNP-encapsulated CPT (NNP-CPT) controls.

C. Immunomodulatory Agents and/or Adjuvants

As demonstrated in the examples, the efficacy of BNP-encapsulated CPT in combination with immune modulation in the form of cytidine-phosphate-guanosine oligodeoxynucleotides (CpGs) was shown to have better than expected benefits.

As used herein an immunomodulatory agent is a substance that stimulates or suppresses the immune system and may help the body fight cancer, infection, or other diseases. Specific immunomodulating agents, such as monoclonal antibodies, cytokines, and vaccines, affect specific parts of the immune system. Non-specific immunomodulating agents, such as BCG and levamisole, affect the immune system in a more general way. As used herein an adjuvant is a non-specific immunostimulatory agent.

Adjuvants commonly work by activating Antigen Presenting Cells (“APCs”) to show T cells that foreign particles are present. Adjuvants also increase recruitment and activation of APCs. APCS are immune cells that engulf foreign particles, digest them into small fragments, and then present the fragments to T cells. Once activated T cells, activate the B cells that produce antibodies. Adjuvants can indirectly activate T cells by releasing complexes called phagosomes, which bind to T cells. The T cells then release cytokines that activate B cells to produce antibodies. This effect enhances the degree of antibody production against a foreign antigen that has entered the animal. Adjuvants also can induce an immune reaction to antigens at specific locations in an organism where the adjuvant is injected. Adjuvants activate the innate immune locally, which draws T cells that are circulating in the blood stream to that location. Adjuvants can control the rate at which antigens are released into the blood stream, for example, in the case of an oil or oil emulsion, delaying release and diffusion away of entrapped agent from a delivery vehicle or particle into the surrounding tissue and into the circulatory system.

Common types of adjuvants include mineral salts, oil emulsions, microbial products, saponins, and some synthetic products. Aluminum salt is a common mineral salt adjuvant. It is good at inducing a Th2 immune response, but is less effective for inducing a Th1 response. The Th2 response results in B cells producing antibodies that neutralize the antigen. The Th1 response results in B cells that produce antibodies that opsonize, or cling to, antigens so that other immune cells can recognize and kill things that are coated with antibodies. Mixtures of oil and water forming emulsions induce strong immune reactions. They are good at inducing a Th2 immune response. They are also good for creating the slow-release effect of antigen depots. Microbial products such as sugars from the cell wall of microbes are foreign particles to animals. These polysaccharide chains can induce a severe immune reaction. Saponins are steroid molecules that have sugar chains attached to them. They naturally occur in plants and some microbes. A low dose can trigger an intense immune response.

Examples of more specific immunostimulatory molecules include TLR ligands, NOD ligands, RLR ligands, CLR ligands, inflammasome inducers, STING ligands, and combinations thereof. Such ligands are known in the art can obtained through commercial vendors such as InvivoGen. Examples of adjuvants include immunostimulants such as TLR ligands (e.g. CpG-containing oligonucleotides, lipopolysaccharide, single-stranded RNA, triphosphate-RNA, double-stranded RNA, imiquimod, resiquimod, polyinosinic:polycytidylic acid, flagellin), RIG-I-like receptors, checkpoint inhibitors (e.g. that target PD-1, PDL-1, CTLA-4, VISTA), immunostimulatory cytokines, bacterial or bacterial components or vaccines, viral or viral components or vaccines, fungi or fungi components or vaccines, colony stimulating factors (e.g. GM-CSF), type I and type II interferons (interferon-alpha, interferon-gamma), interleukins (e.g. interleukin-1, interleukin-12), endogenous immunostimulants (e.g. deoxycholic acid), defective interfering particles (DIPs) of viruses, other pattern recognition receptor (PRRs) agonists/ligands like monophosphoryl lipid A and STING agonists, and other immunostimulants. Ipilimumab and nivolumab type agents

The adjuvant can be an immunostimulatory oligonucleotide or a lipidated immunostimulatory oligonucleotide conjugate thereof, see, e.g., Liu, et al., Nature Letters, 507:519-22 (2014)) (lipo-CpG) and U.S. Pat. No. 9,107,904. In some embodiments, the immunostimulatory oligonucleotide portion of the adjuvant can serve as a ligand for PRRs. Therefore, the oligonucleotide can serve as a ligand for a Toll-like family signaling molecule, such as Toll-Like Receptor 9 (TLR9).

CpG is particularly useful. CpG oligodeoxynucleotides (or CpG ODN) are short single-stranded synthetic DNA molecules that contain a cytosine triphosphate deoxynucleotide (“C”) followed by a guanine triphosphate deoxynucleotide (“G”). The “p” refers to the phosphodiester link between consecutive nucleotides, although some ODN have a modified phosphorothioate (PS) backbone instead. When these CpG motifs are unmethylated, they act as immunostimulants. CpG motifs are considered pathogen-associated molecular patterns (PAMPs) due to their abundance in microbial genomes but their rarity in vertebrate genomes. The CpG PAMP is recognized by the pattern recognition receptor (PRR) Toll-Like Receptor 9 (TLR9), which is constitutively expressed only in B cells and plasmacytoid dendritic cells (pDCs) in humans and other higher primates unmethylated CpG sites can be detected by TLR9 on plasmacytoid dendritic cells and B cells in humans (Zaida, et al., Infection and Immunity, 76(5):2123-2129, (2008)). Therefore, the sequence of the oligonucleotide can include one or more unmethylated cytosine-guanine (CG or CpG, used interchangeably) dinucleotide motifs. The ‘p’ refers to the phosphodiester backbone of DNA, as discussed in more detail below, some oligonucleotides including CG can have a modified backbone, for example a phosphorothioate (PS) backbone.

In some embodiments, an immunostimulatory oligonucleotide can contain more than one CG dinucleotide, arranged either contiguously or separated by intervening nucleotide(s). The CpG motif(s) can be in the interior of the oligonucleotide sequence. Adjuvants acting through TLR9 include DNA of viral or bacterial origin, or synthetic oligodeoxynucleotides (ODN), such as CpG ODN. Numerous nucleotide sequences stimulate TLR9 with variations in the number and location of CG dinucleotide(s), as well as the precise base sequences flanking the CG dimers.

Typically, CG ODNs are classified based on their sequence, secondary structures, and effect on human peripheral blood mononuclear cells (PBMCs). The five classes are Class A (Type D), Class B (Type K), Class C, Class P, and Class S (Vollmer, J & Krieg, A M, Advanced drug delivery reviews 61(3): 195-204 (2009), incorporated herein by reference). CG ODNs can stimulate the production of Type I interferons (e.g., IFNα) and induce the maturation of dendritic cells (DCs). Some classes of ODNs are also strong activators of natural killer (NK) cells through indirect cytokine signaling. Some classes are strong stimulators of human B cell and monocyte maturation (Weiner, G L, PNAS USA 94(20): 10833-7 (1997); Dalpke, A H, Immunology 106(1): 102-12 (2002); Hartmann, G, J of Immun. 164(3):1617-2 (2000), each of which is incorporated herein by reference).

Other PRR Toll-like receptors include TLR3, and TLR7 which may recognize double-stranded RNA, single-stranded and short double-stranded RNAs, respectively, and retinoic acid-inducible gene I (RIG-I)-like receptors, namely RIG-I and melanoma differentiation-associated gene 5 (MDA5), which are best known as RNA-sensing receptors in the cytosol. Therefore, in some embodiments, the oligonucleotide contains a functional ligand for TLR3, TLR7, or RIG-I-like receptors, or combinations thereof.

Additional immunmodulating agents include antibodies to the epidermal growth factor receptor such as panitumumab (VECTIBIX®) and cetuximab (ERBITUX®).

Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)).

Adjuvants can be immunostimulatory oligonucleotides or other immunostimulatory molecules, with or without a lipid tail are provided. Examples of molecules that include a lipid tail, or can be modified to include one, can be, for example, pathogen-associated molecular patterns (PAMPs). PAMPS are recognized by pattern recognition receptors (PRRs). At least five families of PRRs have been shown to initiate pro-inflammatory signaling pathways: Toll-like receptors (TLRs), NOD-like receptors (NLRs), RIG-I-like receptors (RLRs), C-type lectin receptors (CLRs) and cytosolic dsDNA sensors (CDSs). Also, some NLRs are involved in the formation of pro-inflammatory complexes called inflammasomes.

Adjuvants that act through TLR4 include derivatives of lipopolysaccharides such as monophosphoryl lipid A (MPLA; Ribi ImmunoChem Research, Inc., Hamilton, Mont.) and muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland).

Examples of immunostimulatory oligonucleotides, and methods of making them are known in the art, see for example, Bodera, P. Recent Pat Inflamm Allergy Drug Discov. 5(1):87-93 (2011).

In some embodiments, the oligonucleotide includes two or more immunostimulatory sequences.

Microbial cell-wall components such as Pam2CSK4, Pam3CSK4, and flagellin activate TLR2 and TLR5 receptors respectively and can also be used.

Another adjuvant class is phosphorothioate containing molecules such as phosphorothioate nucleotide analogs and nucleic acids containing phosphorothioate backbone linkages.

Adjuvants may also include immunomodulators such as cytokines, interleukins (e.g., IL-1, IL-2, IL-4, IL-5, IL-6, IL-7, IL-12, etc.), interferons (e.g., interferon-gamma), macrophage colony stimulating factor, and tumor necrosis factor.

In a preferred embodiment, the immunomodulatory agents are PD-1 or CTLA4 based drugs, in the class of immune checkpoint modulators. Immune checkpoints can be stimulatory or inhibitory, and tumors can use these checkpoints to protect themselves from immune system attacks. Currently approved checkpoint therapies block inhibitory checkpoint receptors, but investigations into therapies that activate stimulatory checkpoints are also underway. The immune checkpoint modulator can be one that blocks an inhibitory checkpoint or activates a stimulatory checkpoint. Typically, the immune checkpoint modulator is one that induces or otherwise activates or increases an immune response against target cells for example cancer cells or infected cells. In preferred embodiments, the immune checkpoint modulator blocks an inhibitory checkpoint. Blockade of negative feedback signaling to immune cells thus results in an enhanced immune response against tumors. Thus, in some embodiments the immune checkpoint modulator is administered to the subject in an effective amount to block an inhibitory checkpoint. Exemplary compounds are those that block or otherwise inhibit, for example, PD-1, PD-L1, or CTLA4.

### PD-1 Antagonists

Activation of T cells normally depends on an antigen-specific signal following contact of the T cell receptor (TCR) with an antigenic peptide presented via the major histocompatibility complex (MHC) while the extent of this reaction is controlled by positive and negative antigen-independent signals emanating from a variety of co-stimulatory molecules. The latter are commonly members of the CD28/B7 family. Conversely, Programmed Death-1 (PD-1) is a member of the CD28 family of receptors that delivers a negative immune response when induced on T cells. Contact between PD-1 and one of its ligands (B7-H1 or B7-DC) induces an inhibitory response that decreases T cell multiplication and/or the strength and/or duration of a T cell response. Suitable PD-1 antagonists are described in U.S. Pat. Nos. 8,114,845, 8,609,089, and 8,709,416, and include compounds or agents that either bind to and block a ligand of PD-1 to interfere with or inhibit the binding of the ligand to the PD-1 receptor, or bind directly to and block the PD-1 receptor without inducing inhibitory signal transduction through the PD-1 receptor.

In some embodiments, the PD-1 receptor antagonist binds directly to the PD-1 receptor without triggering inhibitory signal transduction and also binds to a ligand of the PD-1 receptor to reduce or inhibit the ligand from triggering signal transduction through the PD-1 receptor. By reducing the number and/or amount of ligands that bind to PD-1 receptor and trigger the transduction of an inhibitory signal, fewer cells are attenuated by the negative signal delivered by PD-1 signal transduction and a more robust immune response can be achieved.

It is believed that PD-1 signaling is driven by binding to a PD-1 ligand (such as B7-H1 or B7-DC) in close proximity to a peptide antigen presented by major histocompatibility complex (MHC) (see, for example, Freeman, Proc. Natl. Acad. Sci. U.S.A, 105:10275-10276 (2008)). Therefore, proteins, antibodies or small molecules that prevent co-ligation of PD-1 and TCR on the T cell membrane are also useful PD-1 antagonists.

In preferred embodiments, the PD-1 receptor antagonists are small molecule antagonists or antibodies that reduce or interfere with PD-1 receptor signal transduction by binding to ligands of PD-1 or to PD-1 itself, especially where co-ligation of PD-1 with TCR does not follow such binding, thereby not triggering inhibitory signal transduction through the PD-1 receptor. Other PD-1 antagonists include antibodies that bind to PD-1 or ligands of PD-1 such as PD-L1 (also known as B7-H1) and PD-L2 (also known as B7-DC), and other antibodies.

Suitable anti-PD-1 antibodies include, but are not limited to, those described in the following publications: PCT/IL03/00425 (Hardy et al., WO/2003/099196), PCT/JP2006/309606 (Korman et al., WO/2006/121168), PCT/US2008/008925 (Li et al., WO/2009/014708), PCT/JP03/08420 (Honjo et al., WO/2004/004771), PCT/JP04/00549 (Honjo et al., WO/2004/072286), PCT/IB2003/006304 (Collins et al., WO/2004/056875), PCT/US2007/088851 (Ahmed et al., WO/2008/083174), PCT/US2006/026046 (Korman et al., WO/2007/005874), PCT/US2008/084923 (Terrett et al., WO/2009/073533), and Berger et al., Clin. Cancer Res., 14:30443051 (2008).

A specific example of an anti-PD-1 antibody is MDX-1106 (see Kosak, US 20070166281 (pub. 19 Jul. 2007) at par. 42), a human anti-PD-1 antibody, preferably administered at a dose of 3 mg/kg.

Exemplary anti-B7-H1 antibodies include, but are not limited to, those described in the following publications: PCT/US06/022423 (WO/2006/133396, pub. 14 Dec. 2006), PCT/US07/088851 (WO/2008/083174, pub. 10 Jul. 2008), and US 2006/0110383 (pub. 25 May 2006). A specific example of an anti-B7-H1 antibody is MDX-1105 (WO/2007/005874, published 11 Jan. 2007)), a human anti-B7-H1 antibody.

For anti-B7-DC antibodies see U.S. Pat. Nos. 7,411,051, 7,052,694, 7,390,888, and U.S. Published Application No. 2006/0099203. The antibody can be a bi-specific antibody that includes an antibody that binds to the PD-1 receptor bridged to an antibody that binds to a ligand of PD-1, such as B7-H1. In some embodiments, the PD-1 binding portion reduces or inhibits signal transduction through the PD-1 receptor.

Other exemplary PD-1 receptor antagonists include, but are not limited to B7-DC polypeptides, including homologs and variants of these, as well as active fragments of any of the foregoing, and fusion proteins that incorporate any of these. In a preferred embodiment, the fusion protein includes the soluble portion of B7-DC coupled to the Fc portion of an antibody, such as human IgG, and does not incorporate all or part of the transmembrane portion of human B7-DC.

The PD-1 antagonist can also be a fragment of a mammalian B7-H1, preferably from mouse or primate, preferably human, wherein the fragment binds to and blocks PD-1 but does not result in inhibitory signal transduction through PD-1. The fragments can also be part of a fusion protein, for example an Ig fusion protein.

Other useful polypeptides PD-1 antagonists include those that bind to the ligands of the PD-1 receptor. These include the PD-1 receptor protein, or soluble fragments thereof, which can bind to the PD-1 ligands, such as B7-H1 or B7-DC, and prevent binding to the endogenous PD-1 receptor, thereby preventing inhibitory signal transduction. B7-H1 has also been shown to bind the protein B7.1 (Butte et al., Immunity, Vol. 27, pp. 111-122, (2007)). Such fragments also include the soluble ECD portion of the PD-1 protein that includes mutations, such as the A99L mutation, that increases binding to the natural ligands (Molnar et al., PNAS, 105:10483-10488 (2008)). B7-1 or soluble fragments thereof, which can bind to the B7-H1 ligand and prevent binding to the endogenous PD-1 receptor, thereby preventing inhibitory signal transduction, are also useful.

PD-1 and B7-H1 anti-sense nucleic acids, both DNA and RNA, as well as siRNA molecules can also be PD-1 antagonists. Such anti-sense molecules prevent expression of PD-1 on T cells as well as production of T cell ligands, such as B7-H1, PD-L1 and/or PD-L2. For example, siRNA (for example, of about 21 nucleotides in length, which is specific for the gene encoding PD-1, or encoding a PD-1 ligand, and which oligonucleotides can be readily purchased commercially) complexed with carriers, such as polyethyleneimine (see Cubillos-Ruiz et al., J. Clin. Invest. 119(8): 2231-2244 (2009), are readily taken up by cells that express PD-1 as well as ligands of PD-1 and reduce expression of these receptors and ligands to achieve a decrease in inhibitory signal transduction in T cells, thereby activating T cells.

Exemplary PD-1 inhibitors include, but are not limited to, Pembrolizumab (formerly MK-3475 or lambrolizumab, Keytruda) was developed by Merck and first approved by the Food and Drug Administration in 2014 for the treatment of melanoma, Nivolumab (Opdivo) was developed by Bristol-Myers Squibb and first approved by the FDA in 2014 for the treatment of melanoma, pidilizumab, by CureTech, AMP-224, by GlaxoSmithKline and MedImmune, AMP-514, by GlaxoSmithKline and MedImmune, PDR001, by Novartis, and cemiplimab, by Regeneron and Sanofi.

Exemplary PD-L1 inhibitors include, but are not limited to, Atezolizumab (Tecentriq) a fully humanised IgG1 (immunoglobulin 1) antibody developed by Roche Genentech. In 2016, the FDA approved atezolizumab for urothelial carcinoma and non-small cell lung cancer, Avelumab (Bavencio) a fully human IgG1 antibody developed by Merck Serono and Pfizer. Avelumab is FDA approved for the treatment of metastatic merkel-cell carcinoma. It failed phase III clinical trials for gastric cancer, Durvalumab (Imfinzi) a fully human IgG1 antibody developed by AstraZeneca. Durvalumab is FDA approved for the treatment of urothelial carcinoma and unresectable non-small cell lung cancer after chemoradiation, BMS-936559, by Bristol-Myers Squibb, and CK-301, by Checkpoint Therapeutics. See, e.g., Iwai, et al., Journal of Biomedical Science, (2017) 24:26, DOI 10.1186/s12929-017-0329-9.

### CTLA4 Antagonists

Other molecules useful in mediating the effects of T cells in an immune response are also contemplated as active agents. For example, in some embodiments, the molecule is an agent binds to an immune response mediating molecule that is not PD-1. In a preferred embodiment, the molecule is an antagonist of CTLA4, for example an antagonistic anti-CTLA4 antibody. An example of an anti-CTLA4 antibody is described in PCT/US2006/043690 (Fischkoff et al., WO/2007/056539).

Dosages for anti-PD-1, anti-B7-H1, and anti-CTLA4 antibody, are known in the art and can be in the range of 0.1 to 100 mg/kg, with shorter ranges of 1 to 50 mg/kg preferred and ranges of 10 to 20 mg/kg being more preferred. An appropriate dose for a human subject is between 5 and 15 mg/kg, with 10 mg/kg of antibody (for example, human anti-PD-1 antibody, like MDX-1106) most preferred.

Specific examples of CTLA antagonists include Ipilimumab, also known as MDX-010 or MDX-101, a human anti-CTLA4 antibody, preferably administered at a dose of about 10 mg/kg, and Tremelimumab a human anti-CTLA4 antibody, preferably administered at a dose of about 15 mg/kg. See also Sammartino, et al., Clinical Kidney Journal, 3(2):135-137 (2010), published online December 2009.

In other embodiments, the antagonist is a small molecule. A series of small organic compounds have been shown to bind to the B7-1 ligand to prevent binding to CTLA4 (see Erbe et al., J. Biol. Chem., 277:7363-7368 (2002). Such small organics could be administered alone or together with an anti-CTLA4 antibody to reduce inhibitory signal transduction of T cells.

### Adjuvants

The adjuvant can also be oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

### III. Methods of Treatment

### A. Disorders to be Treated

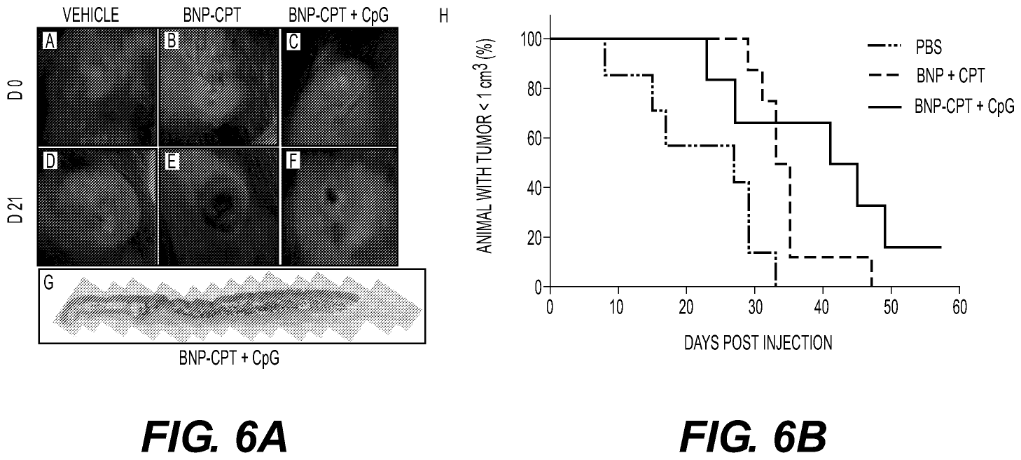

As demonstrated by the examples, co-delivery of an immunostimulatory agent, such as CpG, enhances the capacity of BNP-CPT to eradicate tumors and extend survival by stimulating a local immune response to aid in tumor clearance, as has been well-characterized for CpG stimulation of TLR9 in other cutaneous malignancies, including melanoma (Najar, J. Invest. Dermatol. 128, 2204-2210 (2008); Koster Clin. Cancer Res. 23, 5679-5686 (2017)) and cutaneous T cell lymphoma (CTCL) (Kim J. Am. Acad. Dermatol. 63, 975-983 (2010)). As shown in the examples, through tumor induction and treatment, it was found that mice receiving combination therapy of BNP-CPT plus adjuvant CpG had significantly delayed tumor growth, and full, histologically confirmed resolution in 17% of the SCC tumors (FIG. 6).

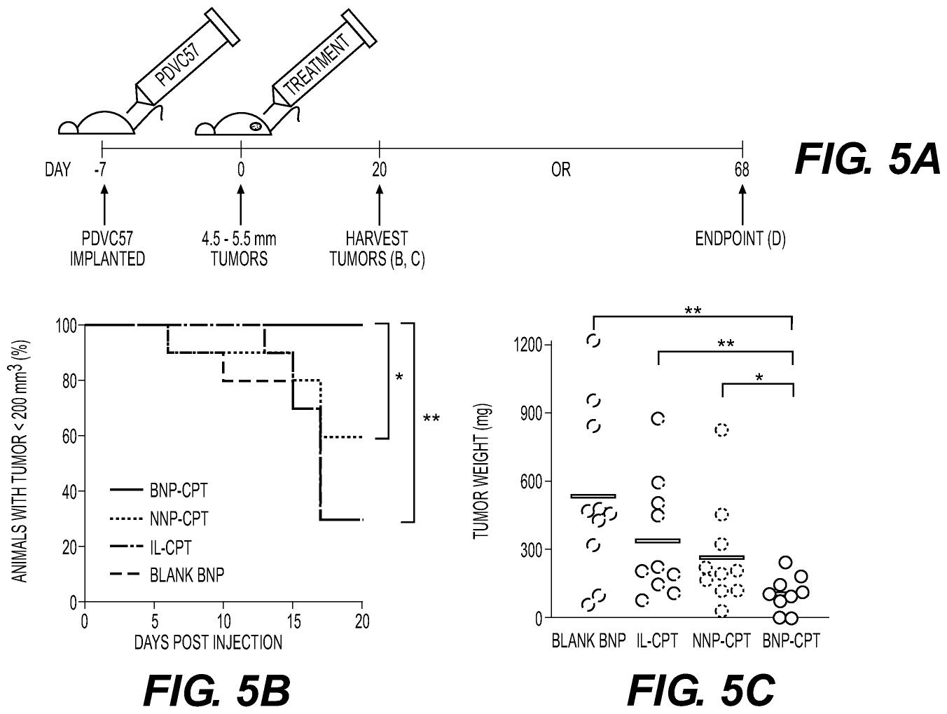

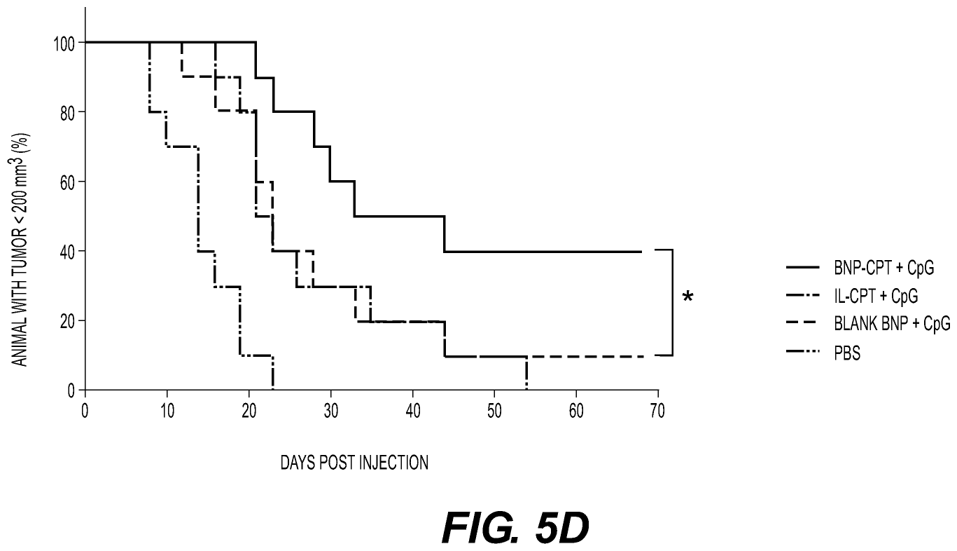

In a separate experiment, mice treated with BNP-CPT plus adjuvant CpG demonstrated significantly delayed tumor growth when compared to mice treated with vehicle, blank BNPs plus adjuvant CpG, or IL-CPT plus adjuvant CpG (FIG. 5D and FIG. 6), and 20% of tumors resolved completely, confirming the therapeutic advantages of BNP delivery.

These data show that local delivery of both chemotherapeutic and immunostimulatory agents represents a practical, nonsurgical approach to the treatment of KDCs.

The examples show that BNPs encapsulating CPT were very effective as a local, nonsurgical treatment for nodular squamous cell carcinoma (SCC) skin cancers in a mouse model. BNP-CPT treatment facilitated tumor destruction and resolution, and was compatible with local immunotherapy. These findings demonstrate that BNP delivery of antitumor agents may provide opportunities for nonsurgical treatment of nodular skin cancers like SCC. Results show that efficacy was more than additive when the BNP delivering chemotherapeutic agent were co-administered with immunostimulatory agents such as CPG.

The examples show that BNPs encapsulating CPT were very effective as a local, nonsurgical treatment for nodular squamous cell carcinoma (SCC) skin cancers in a mouse model. BNP-CPT treatment facilitated tumor destruction and resolution and was compatible with local immunotherapy. These findings demonstrate that BNP delivery of antitumor agents can be effective for nonsurgical treatment of nodular skin cancers like SCC. Results show that efficacy was more than additive when the BNP delivering chemotherapeutic agent were co-administered with immunostimulatory agents such as CPG. Taken together, these results show that percutaneous delivery of a chemotherapeutic agent via BNPs, with or without adjuvant immunostimulation, represents a viable, nonsurgical alternative for treating cutaneous malignancy.

### B. Routes of Administration and Dosing Regimens

The BNPS having encapsulated therein chemotherapeutic agent are administered by injection to a patient in need thereof, typically a patient diagnosed with a cutaneous malignancy. The BNP may be injected alone or in combination with an adjuvant, or the adjuvant administered concurrently or on a different schedule, as needed. In most cases the BNPs will be administered intradermally, subcutaneously and/or intramuscularly in the region associated with the cancer, but can be administered systemically. Dosage will be assessed based on the known dosages for the chemotherapeutic and the adjuvant to have safety and efficacy.

The most common cutaneous malignancies include basal cell carcinoma, squamous cell carcinoma, and melanoma. Primary cutaneous lymphoma, Merkel cell carcinoma, Kaposi sarcoma, adnexal tumor, metastatic cancers to the skin. Cutaneous malignancies vary widely in their aggressiveness, morbidity, and mortality. Basal cell and squamous cell carcinomas are by far the most common cutaneous malignancies. Malignant melanoma is less common and is much more aggressive. This review article covers the recognition, diagnosis, and treatment of less common cutaneous and subcutaneous malignancies. Although they are uncommon, it is important to have the ability to recognize these pathologies. The following malignancies are included: primary cutaneous lymphomas, Merkel cell carcinoma, Kaposi sarcoma, adnexal tumors, and metastatic cancers to the skin.

### Primary Cutaneous Lymphomas

Primary cutaneous lymphomas are a heterogeneous group of non-Hodgkin lymphomas that affect the skin and may progress to systemic disease. These have replaced Hodgkin disease as the most common adult lymphomas, and they are more common among the black population than the white population and are more common in men than in women.

The majority of cases are cutaneous T-cell lymphomas, comprising 65% to 92% of cutaneous lymphomas, of which mycosis fungoides and Sézary syndrome are most common. Mycosis fungoides has more of an indolent behavior, and Sézary syndrome frequently is more aggressive. The tumors of mycosis fungoides can become very large and may be mushroom-shaped, thus the termfungoides. A smaller subset of cutaneous lymphomas includes primary cutaneous CD4⁺ small/medium pleomorphic lymphoma, cutaneous γδ T-cell lymphoma, and cutaneous B-cell lymphoma. The T cells affected are those responsible for skin homing.

The cutaneous lymphomas present a diagnostic challenge, because they can mimic several other dermatologic diseases, including psoriasis, contact dermatitis, nummular eczema, atopic dermatitis, lichen simplex chronicus, lymphoid contact dermatitis, and tinea corporis. Differentiation of cutaneous lymphomas and other conditions is done through historical information, clinical presentation, and histopathologic analysis.

Cutaneous malignancies vary widely in their aggressiveness, morbidity, and mortality. Basal cell and squamous cell carcinomas are by far the most common cutaneous malignancies. Malignant melanoma is less common and is much more aggressive. Although they are uncommon, it is important to have the ability to recognize these pathologies. The following malignancies are included: primary cutaneous lymphomas, Merkel cell carcinoma, Kaposi sarcoma, adnexal tumors, and metastatic cancers to the skin.

### Cutaneous Malignant Neoplasm

Cutaneous malignant neoplasms refers to the broader group of skin growths that are cancerous and may be treated with therapeutic BNPs.

Merkel cell carcinoma (MCC) is a rare and highly aggressive cutaneous neuroendocrine small-cell malignancy. It is highly metastatic to the regional lymphatic basin, as well as nodal and hematogenous spread, and it is often fatal with a 33% mortality rate. MCC also is known as apudoma, primary neuroendocrine carcinoma of the skin, primary small cell carcinoma of the skin, and trabecular carcinoma of the skin. The etiology includes UV solar radiation exposure, immunosuppression, advanced age, and Merkel cell polyomavirus (MCPyV). Kaposi sarcoma (KS) is a rare disease and generally is not believed to be a true malignancy but rather a virally induced angioproliferative disorder associated with human herpesvirus 8 (HHV-8). HHV-8 is required for the development of KS; however, cofactors such as an immunocompromised state also are necessary for KS development. The four subtypes of KS are classic, AIDS-associated, endemic, and immunosuppression-associated. Apocrine and adnexal carcinomas are endocrine mucin-producing tumors that are rare and very commonly are misdiagnosed. This is due to the general infrequency of each of these conditions, as well as similar features to more common skin conditions. Cutaneous metastasis of cancers originating in other organs can occur by hematologic or lymphatic embolization, or direct implantation during surgical procedures.

Many primary tumors have the potential to metastasize to the skin and subcutaneous tissues. These include breast cancer most commonly, but it also can occur with esophageal, gastric, or colon cancers, nasopharyngeal cancer, lymphomas, pancreatic cancer, renal cell carcinoma, lung cancer, and ovarian cancer.

The present invention is further described by the following non-limiting examples.

### Example 1

An in vivo murine model of SCC was established through the intradermal transplantation of PDVC57 SCC cells into syngeneic C57BL/6 mice (Caulin, et al. keratinocytes. Exp. Cell Res. 204, 11-21 (1993)).

To assess BNP retention, tumors injected with Cy5-conjugated NNPs or BNPs and particle distribution visualized via confocal microscopy 72 h after injection.

### Materials and Methods

### Materials

PLA (Mn 12 kDa) was obtained from Lactel Absorbable Polymers. 1,1,1-tris(hydroxymethyl)propane, potassium methoxide, 4-dimethylaminopyridine (DMAP), N,N′-diisopropylcarbodiimide (DIC), dimethylformamide (DMF), diethyl ether (ether), and PO, and poly(Llysine) (PLL) were obtained from Sigma-Aldrich. Glycidol, ethyl acetate (EtOAc), dimethyl sulfoxide (DMSO), dichloromethane (DCM), acetonitrile (ACN), acetone, methanol, deionized water, trifluoroacetic acid (TFA), phosphate-buffered saline (PBS), and DiIC18(5) solid (DiD) were purchased from ThermoFisher Scientific.

Murine endotoxin-free CpG ODN 1826 TLR9 agonist was obtained from InvivoGen (San Diego, CA). Cy5 conjugated PLA was purchased from PolySciTech (product #AV032).

### Methods

PLA-HPG Synthesis. HPG was first synthesized through anionic polymerization. To form the copolymer PLA-HPG, PLA (5 g) and HPG (2.15 g) were dissolved in methylformamide and dichloromethane (DCM), and then dried overnight with 4 Å molecular sieves. Following the dissolution, 0.06 ml N,N′-diisopropylcarbodiimide (DIC) and 10 mg 4-(N,N-dimethylamino)pyridine (DMAP) were added to the mixture, and stirred for 5 days at room temperature. PLA-HPG was then precipitated by adding the mixture to cold ether. The product was then purified by re-dissolving the contents in DCM and re-precipitating with cold ether. This precipitate was washed with cold ether and then lyophilized for 48 hours using the Labconco Freezone Freeze Dry System (Marshall Scientific, Hampton, NH, USA).

NP Preparation. CPT-loaded NNPs were synthesized via the modified single emulsion method (Deng Biomaterials 35, 6595-6602 (2014)). Briefly, 95 mg of PLA-HPG was dissolved in 2.4 mL of ethyl acetate (EtOAc) overnight. Five milligrams of CPT or 0.2 wt % fluorescent dye (DiD) was dissolved in 0.6 mL of dimethyl sulfoxide (DMSO) and was added to the polymer solution, and the combined organic phase was added dropwise to 4 mL of vortexing water. The mixture was further emulsified using a probe-sonicator for four cycles at 10-s intervals. The emulsion was immediately diluted in 10 mL of water with stirring, and ethyl acetate (“EtOAc”) was removed via rotary evaporation at room temperature (RT). NPs were collected via centrifugation at 4,000×g for 30 min at 4° C. using a 100-kDa MWCO centrifugal filter and washed twice with 15 mL of water to isolate NNPs, resulting in a 75% yield. Cy5-conjugated NNPs were prepared by the same method, using the combined solution of 90 mg of PLA-HPG dissolved in 2.4 mL of EtOAc and 10 mg of Cy5-conjugated PLA (AV032; PolySciTech) dissolved in 0.6 mL of DMSO. NNPs were resuspended in 1 mL of water. Oxidative cleavage of terminal vicinal diols was achieved with the procedure of (Deng Proc. Natl. Acad. Sci. U.S.A. 113, 11453-11458 (2016)) as discussed above. NNP stock was diluted threefold with water to approximately 25 mg/mL. One volume of NNPs in water was incubated with 1 vol of 0.1 M NaIO₄ (aq) and 1 vol of 10×PBS on ice for 20 min. The reaction was quenched with 1 vol of 0.2 M Na₂SO_{3(aq)}, and the resulting BNPs were isolated using a 100-kDa MWCO centrifugal filter. BNPs were washed twice with 15 mL of water.

CPT Quantification. Drug incorporation efficiency was determined through the quantification of CPT by exploiting the intrinsic fluorescence of CPT under acidic conditions, as described by (Liu, Biomaterials 30, 5707-5719 (2009)). Briefly, lyophilized NP were dissolved in DMSO. Ten microliters of NP were mixed with 10 μL of 1 N HCl, 10 μL of 10% SDS, and 1 mL of PBS, in that order. A standard curve was similarly prepared with known concentrations of CPT in DMSO. To quantify CPT in NP in suspension, the same assay was performed using NP (in dH₂O) diluted in DMSO at a 1:50 dH₂O:DMSO (vol/vol) ratio, and a standard curve prepared with known concentrations of CPT in a 1:50 dH₂O:DMSO (vol/vol) vehicle. In both cases, the fluorescence of CPT was measured using a Spec-Q: 11 traMax M5 microplate reader (Molecular Devices; excitation [Ex]/emission [Em], 370/428) and compared to CPT standards to quantify CPT concentration and drug loading efficiency. The results of a comparison of the two methods are shown below.

TABLE S2
Comparison of CPT quantification within particles
in suspension or within lyophilized particles.

| NP | Suspension Method | Lyophilized Method |
|---|---|---|
| Batch (n = 3) | CPT mg/mL | CPT mg/mL |
| NNP-CPT (1) | 2.05 | 2.00 |
| BNP-CPT (1) | 2.94 | 2.81 |
| NNP-CPT (2) | 1.13 | 1.00 |
| Bnp-CPT (2) | 1.06 | 1.05 |
| NNP-CPT (3) | 2.01 | 1.97 |
| BNP-CPT (3) | 1.84 | 1.90 |

### Transmission Electron Microscopy (TEM) of NPs.

Following fabrication and conversion, a single drop of 1 mg/mL NP suspension of NNP-CPT and BNP-CPT were applied on charged, carbon-coated copper grids (Electron Microscopy Sciences). The droplet was then blotted with a piece of filter paper. Following a minute of drying, a droplet of uranyl acetate was applied for 30 seconds, before submerging the copper grid in a droplet of water. The samples were then mounted for imaging with TEM (FEI Tecnai Osiris, Yale Institute for Nanoscience and Quantum Engineering) operated at accelerating voltage of 200 kV.

### Drug Release Kinetics.

To assess the rate of drug release from both NNP-CPT and BNP-CPT, 0.15 mg of NNP-CPT and BNP-CPT (100 μL of 1.5 mg/mL NPs with 5% CPT loading) were dispersed within 1.0 mL of 1×PBS at 37° C. using Slide-A-Lyzer™ mini-dialysis devices (ThermoFisher) in triplicates. The dialysis tubes were then placed on shaking racks at 37° C., and at specific intervals (3, 6, 9, 18, 24, 48, 72, 96, 120, 144 hours), the solution was collected and replaced with fresh 1×PBS. The CPT content in the dialyzed fluid was then quantified using the SpectraMax M5 microplate reader (Molecular Devices, Ex/Em: 370/428) and a CPT standard curve, as described above.

### NP Bioadhesiveness

Glass slides were coated with 0.01% poly-l-lysine (PLL, Sigma-Aldrich) in ddH₂O and allowed to dry overnight. PLL-coated slides were then incubated with 60 μL of either NNP-CPT or BNP-CPT suspended in ddH₂O (50 mg/mL). Following a 30 min incubation, slides were washed with ddH₂O for 5 min at 300 RPM on an orbital plate shaker. After decanting the remaining liquid, 500 μL of DMSO was added to each slide and the remaining CPT-loaded NPs were harvested from the slide. The concentration of remaining CPT on each slide was quantified as described in the methods of the main text. Furthermore, the concentration of bioadhesive particles remaining on each slide following washing was calculated using the remaining CPT value and drug incorporation efficiency of each particle lot. The NP retention was normalized to each respective group's unwashed control slides.

### In Vitro Tumor Cell Binding and Uptake of NP.

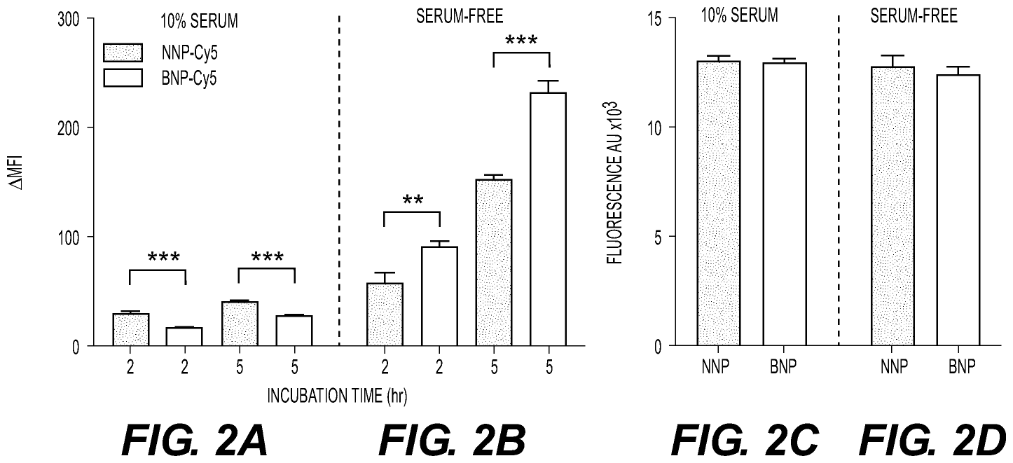

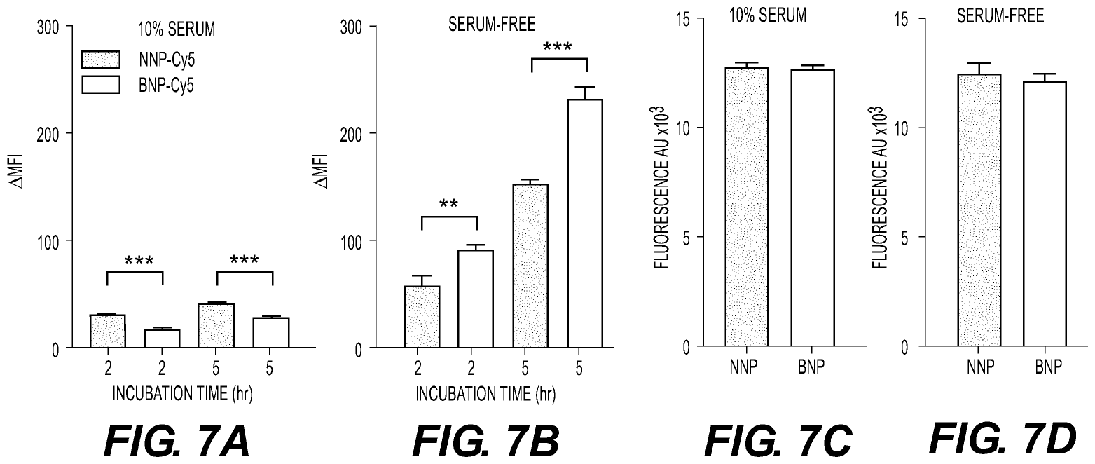

PDV cells were harvested using 0.25% Trypsin-EDTA and washed with HBSS containing 10% FBS, 1% 10,000 U/ml-μg/ml penicillin-streptomycin (Pen/Strep), and 10 mM Hepes. Cells were resuspended in CRPMI (RPMI1640 plus 10% FBS, 10 mM Hepes, 1% non-essential amino-acids, 2 mM L-glutamine, 1 mM sodium pyruvate, 0.05 mM 0-mercaptoethanol, and 1% Pen/Strep), counted, plated at a density of 30,000 cells per well in 24-well plates and cultured overnight. Following overnight culture, the media was aspirated and replaced with medium containing either NNP-Cy5, BNP-Cy5, NNP-DID or BNP-DID (1 mg/ml NP) and cultured as indicated in each experiment, with cells harvested at various timepoints for analysis using either flow cytometry or confocal microscopy. In experiment FIG. 2B and FIG. 7 cells were cultured with NP in CRPMI prepared without added FBS (serum-free CRPMI).

At harvest, the wells were washed 5 times with 1×PBS before the cells were detached with Accutase. For flow cytometric analysis, cells were fixed in 1% PFA, washed in PBS containing 2% FBS, and held at 4° C. Data was collected using the Stratedigm S1000EX (Stratedigm Inc.) and analyzed with FlowJo v 10.4 (FlowJo LLC, Ashland, OR, USA). Baseline fluorescence of NNP-Cy5, BNP-Cy5, NNP-DID and BNP-DID solutions were assessed using a Promega GloMax with red filter kit (Ex 625 nm/Em 660-720 nm).

For confocal microscopy, cells were washed with stain buffer (1×PBS, 1% FBS, 0.09% NaN3) and blocked for 15 min on ice using anti-FcR (clone 2.4G2, 5 μg/ml, BD Biosciences), normal hamster IgG and normal rat IgG (10 μg/ml each, Jackson Immunoresearch). Cells were then stained for 25 min on ice with FITC anti-mouse CD326 Ep-CAM (2.5 μg/ml, BIOLEGEND™) followed by ALEXA™-488-anti-FITC (5 μg/ml, ThermoFisher). After washing, cells were fixed in 1% PFA for 20 minutes, washed with PBS, spun onto slides using the CYTOSPIN™ 4 Cytocentrifuge (Thermo Fischer Scientific) at 800 rpm for 5 minutes and mounted using PROLONG GOLD ANTIFADE MOUNTANT™ with DAPI (ThermoFisher). Z-stacked images were collected using a Leica SP5 confocal microscope and images were processed with Fiji/ImageJ (Rogers, JAMA Dermtol. 151, 1081-1086 (2015)).

### Assessment of Endocytic Mechanisms of NP Uptake.

PDV cells were plated at a Q: 12 density of 30,000 cells per well in 24-well plates in complete RPMI (CRPMI) and allowed to adhere. The media was then aspirated and replaced with CRPMI containing either NNP-Cy5 or BNP-Cy5 (1 mg/mL NP) for 16-h culture (37° C., 5% CO₂). For the final 6 h of culture, vehicle (DMSO; 0.1%), chloropromazine (CPZ) (20 μM; Sigma), an inhibitor of clathrin-dependent endocytosis, or 5-(N-ethyl-N-isopropyl)-amiloride (EIPA) (50 μM; Sigma), an inhibitor of macropinocytosis, were added to the wells. At harvest, the wells were washed five times with 1×PBS before the cells were detached with Accutase. After a brief fixation in 1% PFA, cell-associated NP fluorescence was assessed by flow cytometry using STRATEDIGM™ S1000EX (STRATEDIGM), and data were analyzed using FLOWJO™ v 10.4 (FLOWJO). Baseline fluorescence of NNP-Cy5 and BNP-Cy5 solutions was assessed using a PROMEGA GLOMAX™ with red filter kit (Ex, 625 nm/Em, 660 to 720 nm).

### In Vitro Cytotoxicity Assays.

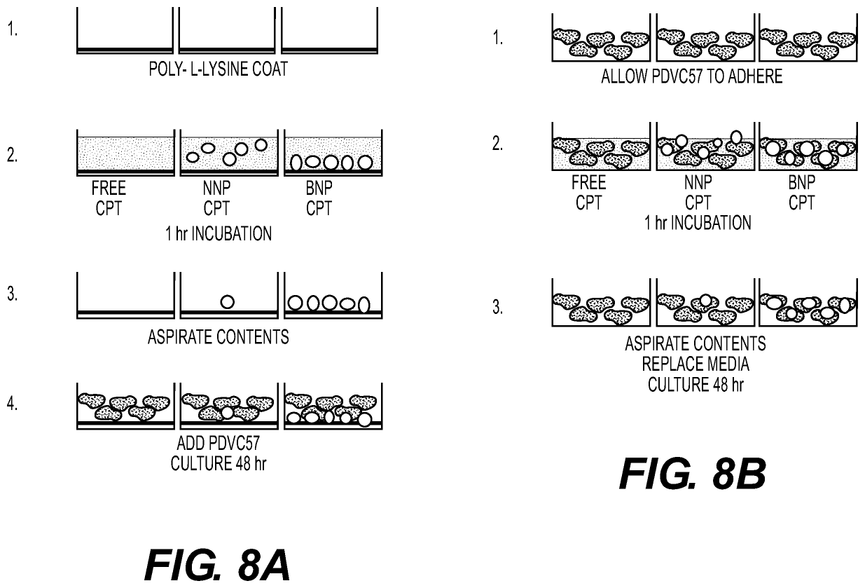

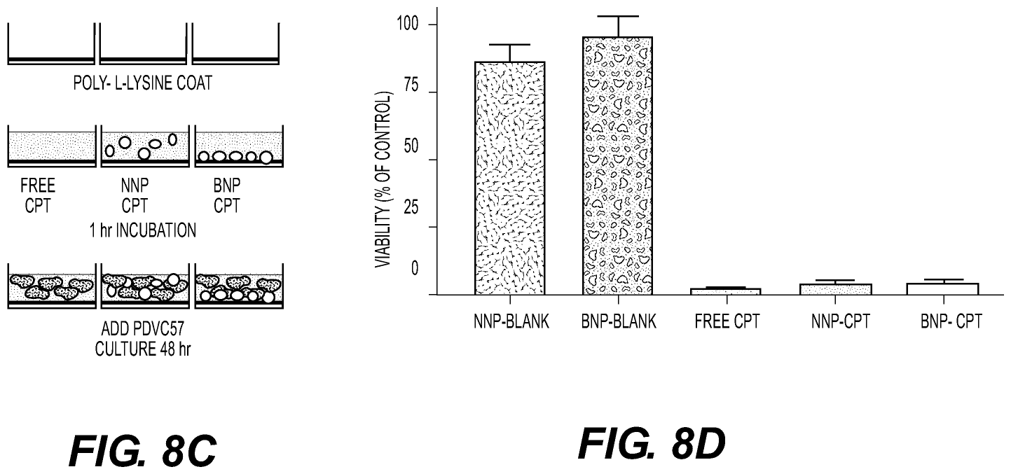

Cytotoxicity assays were conducted in using two methods. In the first method (FIG. 8A, 8C), 96-well tissue culture plates (Thermo Scientific, Waltham, MA, USA) were coated with 0.01% poly-l-lysine (Sigma Aldrich, St. Louis, MO, USA) in ddH₂O and allowed to dry overnight. Solutions of free CPT dissolved in DMSO, NNP-CPT, and BNP-CPT with CPT concentrations of 5 μM were then prepared in ddH₂O and added to the poly-1-lysine coated wells. Following a 1 hr incubation, the solutions were aspirated. During the incubation, PDVC57 cells were harvested, resuspended at 100,000 cells/mL in CRPMI and 100 μl per well added following aspiration of the CPT or NP-CPT solutions.

In the second method (FIG. 8B), PDVC57 cells were harvested, resuspended in CRPMI and plated at a density of 5,000 cells per well in a flat-bottom 96-well plate. 12 hours after plating, the prepared solutions of 5 μM CPT, NNP-CPT, BNP-CPT, and vehicle controls in CRPMI were added to the wells. Following a 1 hr. incubation, the solutions were aspirated and replaced with 100 μl of CRPMI. In both methods, viability was assessed after an additional 48 hr culture using the CELLTITER-GLO® Luminescent Cell Viability Assay (Promega) and Victor Light Luminescence Counter (Perkin Elmer). Luminescence values for wells treated with free CPT were normalized to a vehicle control consisting of CRPMI with 3% DMSO, the concentration of DMSO used in the free CPT samples, while the luminescence of wells treated with NNP-CPT or BNP-CPT were normalized to a vehicle control of CRPMI with 2.3% PBS, the highest concentration of PBS used in the NNP-CPT and BNP-CPT samples.

FIG. 8A shows a simulated, protein-rich tumor matrix created by coating a 96 well-plate with 0.01% poly-l-lysine in 1×PBS. Blank NNPs, blank BNPs, free CPT, NNP-CPT, or BNP-CPT (5 μM CPT) were added to the wells for an hour-long incubation, and then carefully aspirated. Following aspiration, PDVC57 cells were plated on the pre-treated wells (5000 cells/well) in CRPMI, and incubated for 48 hours. After 48 hours, cell viability was assessed using Cell-Titer Glo. (8D) PDVC7 cells were harvested and seeded on a 96-well plate (5000 cells/well). Free CPT, NNP-CPT, or BNP-CPT (5 μM CPT) were added to the wells for an hour-long incubation, then carefully aspirated and the media replaced. FIG. 8B is a control for assay (8A) in which there is no poly-l-lysine attached to the plate. FIG. 8C shows the poly-l-lysine coated plates incubated with blank (unloaded) NP, free CPT, NNP-CPT or BNP-CPT, but these solutions are not aspirated before the addition of PDVC57 cells. FIG. 8D is a graph of the results of viability testing, showing the control assay demonstrates comparable killing of PDVC57 by free CPT, NNP-CPT and BNP-CPT, and that there is no toxicity associated with unloaded NNP-blank or BNP-blank particles.

### Tumor Induction.

All animal procedures were conducted in accordance with Yale Institutional Animal Care and Use Committee guidelines. Mice were housed in the Yale Animal Resource Center and afforded free access to food and water. Wild-type C57BL/6J mice were obtained from The Jackson Laboratory. To prepare the animals for tumor transplantation, the dorsal right flank was shaved using electric clippers. PDVC57 cells were harvested, washed, and resuspended in 1×PBS on ice at a concentration of 5.0×10⁷ cells per mL. Mice were then anesthetized in an induction chamber with 30% isoflurane in propylene glycol and the dorsal right flank injected intradermally/subcutaneously with 5.0×10⁶ cells in 100 μl using a 27-gauge syringe, forming a small bleb at the site of injection.

### Intratumoral NP Retention Assessed by Confocal Microsopy.

Wild type C57Bl/6J mice were obtained from The Jackson Laboratory (Bar Harbor, ME, USA). PDVC57 SCC tumors were transplanted with a subcutaneous injection of five million cells. When the tumors reached a diameter of 5 mm, they were injected with either NNP-Cy5 or BNP-Cy5 (5 mg NP/50 μL). Before injection, both NNP-Cy5 and BNP-Cy5 were vortexed and sonicated using a VIBRA CELL SONICATOR™ (Sonics & Materials Inc., Danbury, CT, USA) for 30 seconds. Tumors were harvested three days following injection, bisected, frozen in Tissue-Tek OCT compound (Electron Microscopy Sciences) on dry ice and stored at −80° C. for cryosectioning.

Frozen sections were mounted on glass slides using PROLONG GOLD ANTIFADE™ with DAPI. Confocal microscopy of the injected tumor samples was conducted using a Zeiss 880 AIRYSCAN™ and captured images were processed using Fiji/ImageJ (Rogers, JAMA Dermtol. 151, 1081-1086 (2015)).

Incidence estimate of nonmelanoma skin cancer (keratinocyte carcinomas) in the U.S. population, 2012. JAMA Dermatol. 151, 1081-1086 (2015).

### CPT Quantification.

Drug incorporation efficiency was determined through the quantification of CPT by exploiting the intrinsic fluorescence of CPT under acidic conditions. Briefly, lyophilized NP were dissolved in DMSO. Ten microliters of NP were mixed with 10 μL of 1 N HCl, 10 μL of 10% SDS, and 1 mL of PBS, in that order. A standard curve was similarly prepared with known concentrations of CPT in DMSO. To quantify CPT in NP in suspension, the same assay was performed using NP (in dH₂O) diluted in DMSO at a 1:50 dH₂O:DMSO (vol/vol) ratio, and a standard curve prepared with known concentrations of CPT in a 1:50 dH2O:DMSO (vol/vol) vehicle. In both cases, the fluorescence of CPT was measured using a Spec-Q: 11 traMax M5 microplate reader (Molecular Devices; excitation [Ex]/emission [Em], 370/428) and compared to CPT standards to quantify CPT concentration and drug loading efficiency. The results of a comparison of the two methods are shown below:

TABLE S2
Comparison of CPT quantification within particles
in suspension or within lyophilized particles.

| NP | Suspension Method | Lyophilized Method |
|---|---|---|
| Batch (n = 3) | CPT mg/mL | CPT mg/mL |
| NNP-CPT (1) | 2.05 | 2.00 |
| BNP-CPT (1) | 2.94 | 2.81 |
| NNP-CPT (2) | 1.13 | 1.00 |
| Bnp-CPT (2) | 1.06 | 1.05 |
| NNP-CPT (3) | 2.01 | 1.97 |
| BNP-CPT (3) | 1.84 | 1.90 |

### Assessment of Endocytic Mechanisms of NP Uptake.

PDV cells were plated at a Q: 12 density of 30,000 cells per well in 24-well plates in complete RPMI (CRPMI) and allowed to adhere. The media was then aspirated and replaced with CRPMI containing either NNP-Cy5 or BNP-Cy5 (1 mg/mL NP) for 16-h culture (37° C., 5% C02). For the final 6 h of culture, vehicle (DMSO; 0.1%), chloropromazine (CPZ) (20 μM; Sigma), an inhibitor of clathrin-dependent endocytosis, or 5-(N-ethyl-N-isopropyl)-amiloride (EIPA) (50 μM; Sigma), an inhibitor of macropinocytosis, were added to the wells. At harvest, the wells were washed five times with 1×PBS before the cells were detached with ACCUTASE™. After a brief fixation in 1% PFA, cell-associated NP fluorescence was assessed by flow cytometry using STRATEDIGM™ S1000EX (STRATEDIGM), and data were analyzed using FLOWJO™ v 10.4 (FLOWJO). Baseline fluorescence of NNP-Cy5 and BNP-Cy5 solutions was assessed using a PROMEGA GLOMAX™ with red filter kit (Ex, 625 nm/Em, 660 to 720 nm).

### Intratumoral NP Retention.

When tumors reached a diameter of about 5 mm, they were treated with either free CPT (0.25 mg of CPT/50 μL of intralipid), NNP-CPT (0.25 mg of CPT/50 μL of PBS), or BNP-CPT (0.25 mg of CPT/50 μL of PBS). Following injection, tumors were harvested and lyophilized at 0, 48, and 240 h. Lyophilized tumors were then homogenized with the PRECELLYS™ 24 Homogenizer with soft tissue homogenizing 1.4-mm ceramic beads (Bertin) for 4 min and 35 s in 45-s pulses with 500 μL of DMSO. The first 500 μL was removed and another 500 μL added for another round of homogenization. The homogenate was centrifuged at 10,000×g for 5 min to pellet residual tumor and debris, the supernatant removed, and CPT quantified using its intrinsic fluorescence as described above.

### In Vivo Assessment of CPT NP Efficacy.

PDVC57 tumors were induced as described above. When tumors attained a diameter of approximately 5 mm, they were injected with either blank BNPs (1.99 mg/50 μL of PBS), IL-CPT (0.25 mg of CPT/50 μL), NNP-CPT (0.25 mg of CPT/50 μL of PBS), or BNP-CPT (0.25 mg of CPT/50 μL of PBS) using a 27-gauge needle, which was positioned in the center of the tumor. The entire treatment volume was administered intratumorally over 15 s. The needle was then held in this position for 10 s and withdrawn slowly in order to prevent leakage of drug from the injection site. In vivo dosing was based on pilot studies that revealed antitumor activity (without clinical evidence of systemic toxicity) when a single dose of 0.25 mg of CPT was delivered by intratumoral injection.

Statistics. The Mantel-Cox test was used to assess Kaplan-Meier curves (FIGS. 5 B and D). All other experimental groups were compared using an unpaired one-tailed Student t test with significance established at P<0.05. All statistical analyses were performed using GraphPad Prism 8.3 software.

### Results

### BNP-CPT Synthesis and Characterization.

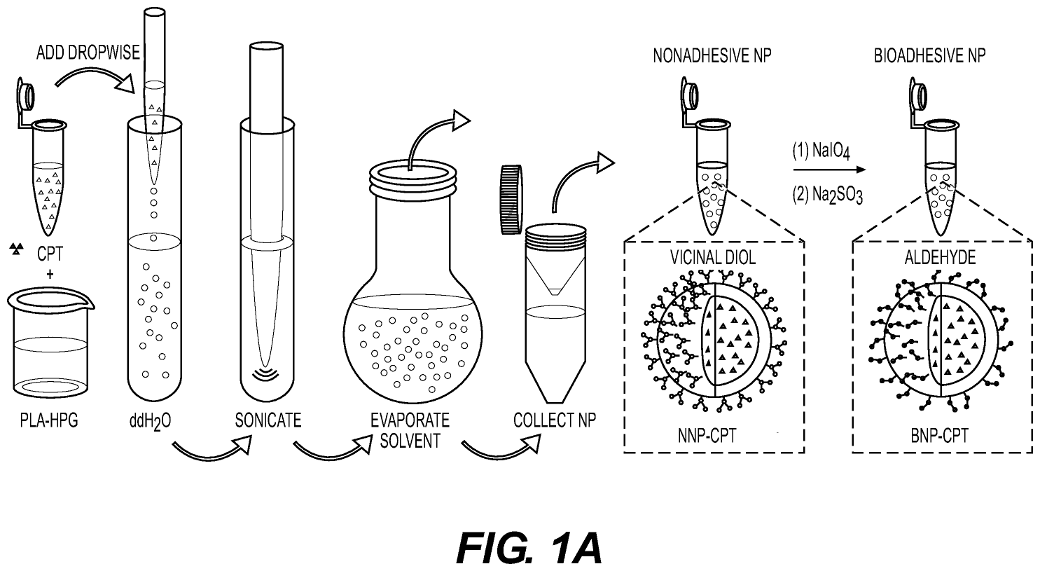

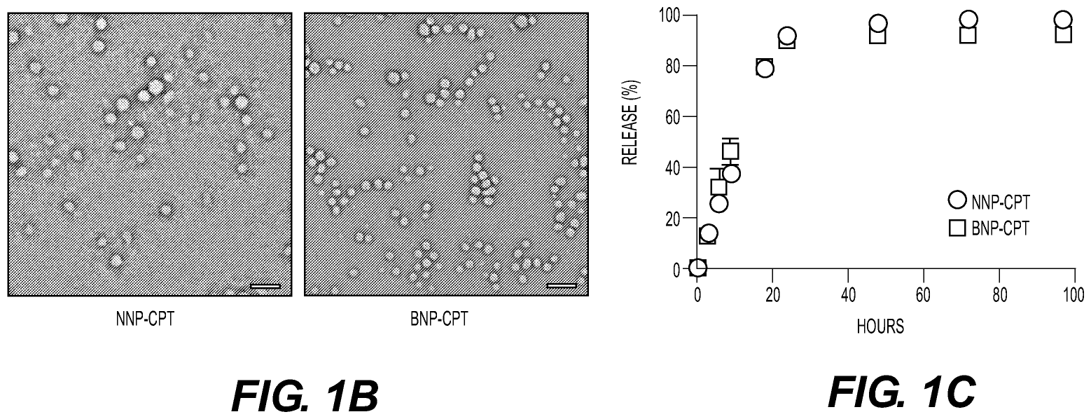

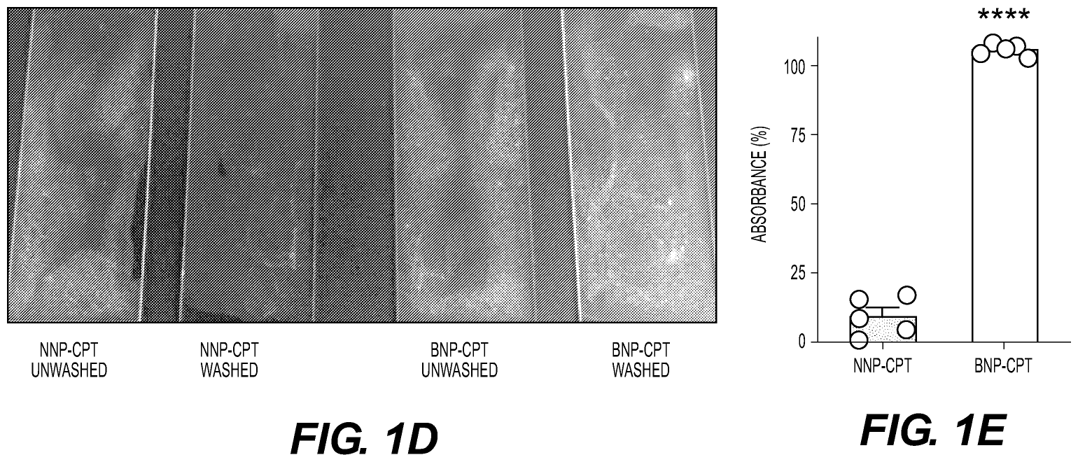

After formation of NNPCPT, and their subsequent modification to BNP-CPT (FIG. 1A), both NNP-CPT and BNP-CPT demonstrated an average size distribution between 200 and 300 nm by dynamic light scattering, demonstrating consistent conversion without significant changes to the hydrodynamic size, as shown below:

TABLE S2
Comparison of CPT quantification within particles
in suspension or within lyophilized particles.

| NP | Suspension Method | Lyophilized Method |
|---|---|---|
| Batch (n = 3) | CPT mg/mL | CPT mg/mL |
| NNP-CPT (1) | 2.05 | 2.00 |
| BNP-CPT (1) | 2.94 | 2.81 |
| NNP-CPT (2) | 1.13 | 1.00 |
| Bnp-CPT (2) | 1.06 | 1.05 |
| NNP-CPT (3) | 2.01 | 1.97 |
| BNP-CPT (3) | 1.84 | 1.90 |

The largely uniform and spherical structure of these PLA-HPG NPs was confirmed using transmission electron microscopy, and that the oxidative conversion of NNP-CPT to BNP-CPT did not negatively alter the morphology (FIG. 1B).

### In Vitro Kinetics of Drug Release

Release kinetics were observed at physiological temperature (37° C.). Sustained and maximal (˜90%) release from both NNPs and BNPs over 24 h (FIG. 1C). To simulate the protein-rich tumor microenvironment, poly-L-lysine-coated slides were prepared, incubated with aqueous suspensions of either NNP-CPT or BNP-CPT, and the relative adherence quantified on the amount of CPT recovered from the slides after washing (FIG. 1D). Virtually all of the CPT was recovered when encapsulated in BNPs, compared to an approximately 10% recovery rate when encapsulated within NNPs (FIG. 1E).

To assess whether the increased bioadhesion from BNP conversion results in greater association with tumor cells, murine SCC cells (PDVC57) were incubated with NNPs or BNPs incorporating a fluorescent dye (DiD). By confocal microscopy, extensive adhesion of BNPs around and within the SCC cells relative to dye-containing NNP was observed. The binding affinity of BNP-CPT was further assessed by incubating PDVC57 cells with Cy5-conjugated NPs or DiD-loaded NPs prior to measuring cellular association as a function of time via flow cytometry. Because BNP may bind serum proteins found in cell culture media, and this could affect NP association with tumor cells, NNP-Cy5 and BNPCy5 tumor cell association following short-term culture in serum versus serum-free media was compared.

BNPs readily associate with tumor cells, shown by graphs comparing NP association with tumor cells following short (up to 5 h) incubation in serum-containing vs. serum-free conditions (2A, 2B). Baseline fluorescence of both dye-conjugated (Cy5) and -loaded (DiD) NNP and BNP before incubation is similar. Cellular association of Cy5-NPs (Left) and DiD-NPs (Right) with extended incubation in serum containing media shows that even with prolonged exposure, BNPs associate with tumor cells more strongly than NNPs (2C, 2D).

In serum free media (FIG. 2B and FIG. 2D), BNPs associated with tumor cells to a greater extent than NNPs, and this was most pronounced at later time points (12 to 24 h). In serum-containing media (FIGS. 2C and 2D), at early time points (up to 5 h) no BNP advantage was seen; however, with extended culture (24 to 72 h), BNPs show significantly greater tumor cell association than NNPs. This was true regardless of whether the dye was covalently conjugated to the polymer (Cy5) or encapsulated within (DiD) the particles. BNP binding to serum proteins may initially slow tumor cell uptake in vitro. Alternatively, cells may respond to the absence of serum by activating additional uptake mechanisms. Because extracellular proteins may also play a role in vivo, serum containing media was used for the following experiments designed to investigate NP uptake mechanisms.

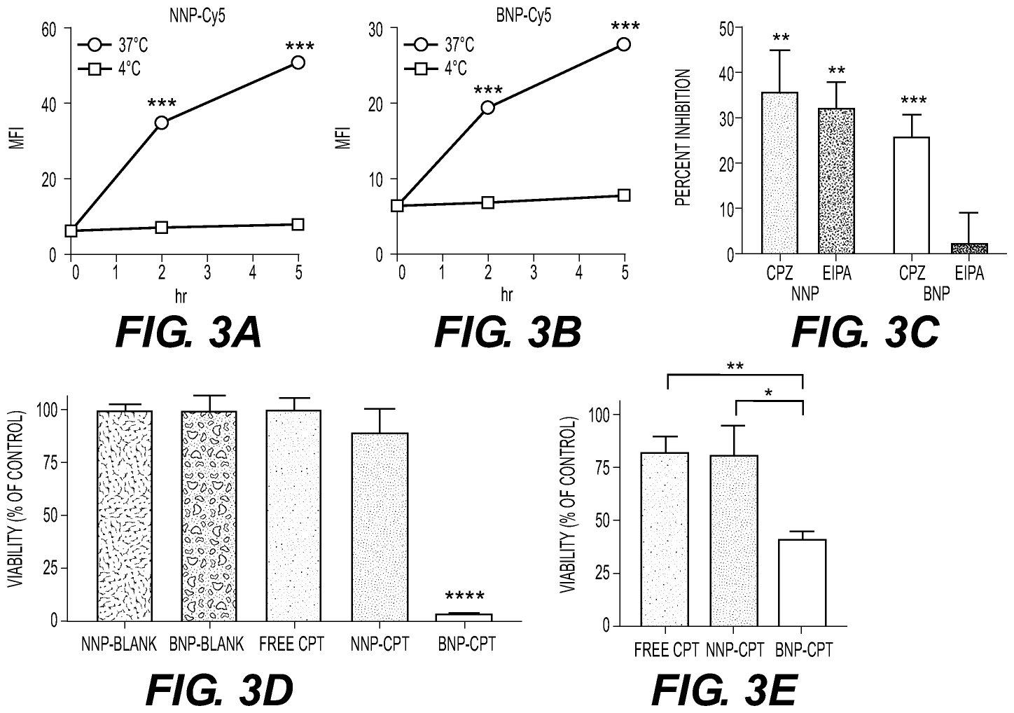

FIG. 3A-3D show that BNPs facilitate both active internalization by tumor cells and binding to extracellular proteins to promote cytotoxicity. Cellular association or uptake of NPs occurs via an active process given that tumor cell incubation with NNP or BNP a 4° C. results in significantly reduced cellular association as compared to 37° C. (FIG. 3A). Either clathrin mediated endocytosis or macropinocytosis was selectively blocked by applying drugs during incubation with BNPs or NNPs. A significant decrease in NNP uptake after blocking either clathrin-mediated endocytosis or macropinocytosis was observed (FIG. 3B). In contrast, only interruption of clathrin-mediated endocytosis resulted in a significant decrease in uptake of BNPs in this assay. Overall, these data indicate that BNPs may preferentially utilize clathrin mediated entry, while NNPs display more promiscuity in cellular entry. Extracellular Proteins Facilitate Tumor Cell Killing by BNP-CPT. To assess the advantages of bioadhesion, further in vitro cytotoxicity studies in a simulated tumor matrix were conducted (FIG. 2F). After precoating 96-well plates with poly-L-lysine, the plates were incubated with either free CPT, NNPCPT, BNP-CPT, or unloaded NPs (NNP-blank, BNP-blank) for an hour before aspirating the contents and seeding PDVC57 tumor cells into the pretreated wells.

### Extracellular Proteins Facilitate Tumor Cell Killing by BNP-CPT.

To assess the advantages of bioadhesion, further in vitro cytotoxicity studies were conducted in a simulated tumor matrix. After precoating 96-well plates with poly-L-lysine, the plates were incubated with either free CPT, NNPCPT, BNP-CPT, or unloaded NPs (NNP-blank, BNP-blank) for an hour before aspirating the contents and seeding PDVC57 tumor cells into the pretreated wells. After an additional 48-h incubation, it was found that BNP-CPT exhibited significantly greater cellular toxicity, when compared to free CPT or NNPCPT (FIG. 3C). Neither unloaded NNPs or unloaded BNPs demonstrated any tumor cell cytotoxicity, indicating that all BNP antitumor effects were attributable to the CPT provided by the NPs (FIG. 3C). Furthermore, these results were recapitulated in a modified experiment in which PDVC57 cells were first seeded, and then incubated with either free CPT, NNP-CPT, or BNPCPT for an hour, before the treatment volume was aspirated. After a 48-h incubation, cell viability was assessed, with BNP-CPT exerting greater cellular toxicity relative to controls (FIG. 3D).

Cellular association or uptake of NPs occurs via an active process given that tumor cell incubation with NNP or BNP at 4° C. results in significantly reduced cellular association as compared to 37° C. (FIG. 3A). Either clathrin mediated endocytosis or macropinocytosis was selectively blocked by applying drugs during incubation with BNPs or NNPs. A significant decrease in NNP uptake after blocking either clathrin-mediated endocytosis or macropinocytosis was observed (FIG. 3B). In contrast, only interruption of clathrin-mediated endocytosis resulted in a significant decrease in uptake of BNPs in this assay. Overall, these data indicate that BNPs may preferentially utilize clathrin mediated entry, while NNPs display more promiscuity in cellular entry.

A simulated, protein-rich tumor matrix was created by coating a 96 wellplate with 0.01% poly-l-lysine in 1×PBS. Blank NNPs, blank BNPs, free CPT, NNP-CPT, or BNP-CPT (5 μM CPT) were added to the wells for an hour-long incubation, and then carefully aspirated. Following aspiration, PDVC57 cells were plated on the pre-treated wells (5000 cells/well) in CRPMI, and incubated for 48 hours. After 48 hours, cell viability was assessed using Cell-Titer Glo. PDVC7 cells were harvested and seeded on a 96-well plate (5000 cells/well). Free CPT, NNP-CPT, or BNP-CPT (5 μM CPT) were added to the wells for an hour-long incubation, then carefully aspirated and the media replaced. After 48 hours, cell viability was assessed using Cell-Titer Glo. A control for assay in which the poly-l-lysine coated plates are incubated with blank (unloaded) NP, free CPT, NNP-CPT or BNP-CPT, but these solutions are not aspirated before the addition of PDVC57 cells. Results of the control assay demonstrates comparable killing of PDVC57 by free CPT, NNP-CPT and BNP-CPT, and also shows that there is no toxicity associated with unloaded NNP-blank or BNPblank particles.

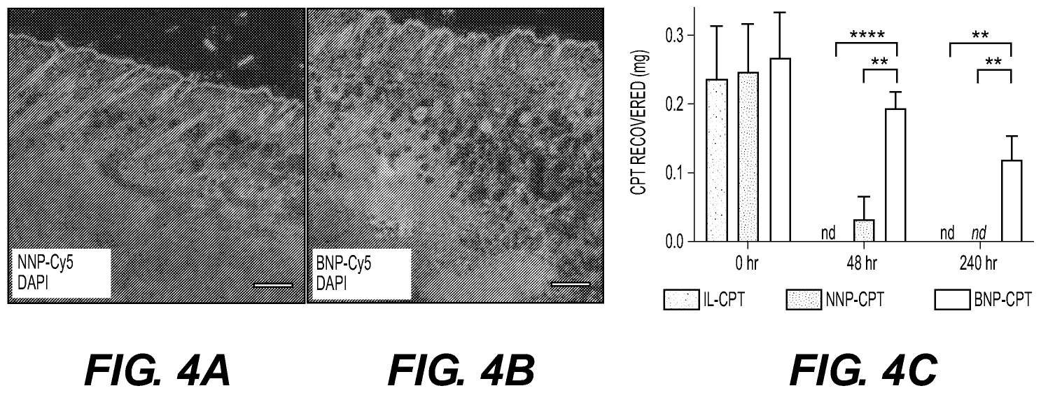

NNPs demonstrated a punctate pattern of distribution with poor intratumoral retention proximal to injection site (FIG. 4A), while BNPs revealed a sustained distribution and higher retention, with an intercalating appearance within the tumor parenchyma (FIG. 4B). An independent set of tumors was then injected with equal doses of CPT, either as free drug suspended in intralipid (IL-CPT), or incorporated as NNP-CPT or BNP-CPT, and harvested the tumors 48 and 240 h later to assess CPT tumor retention.

The vast majority of CPT injected (72%) was recovered from the BNP-CPT-treated mice 48 h after injection, but no CPT was recovered from ILCPT-injected mice at that same time point (FIG. 4C). Furthermore, 240 h following injection, 45% of the CPT from the BNPCPT-injected mice was recovered in the tumor, as compared to the undetectable levels of free CPT and NNP-CPT (FIG. 4C). Taken together, these data indicate that incorporation of CPT into BNPs markedly improves retention within tumors for up to 10 d after injection, via mechanisms that increase tumor cell binding, tumor cell uptake, and persistence within the tumor microenvironment.

### Example 2: BNP-CPT Increases Tumor Retention and Antitumor Efficacy in a Preclinical Model, Especially in Combination with Adjuvant

### Materials and Methods

### In Vivo Assessment of CPT NP Efficacy

PDVC57 tumors were induced as described above. When tumors attained a diameter of approximately 5 mm, they were injected with either blank BNPs (1.99 mg/50 μL of PBS), IL-CPT (0.25 mg of CPT/50 μL), NNP-CPT (0.25 mg of CPT/50 μL of PBS), or BNP-CPT (0.25 mg of CPT/50 μL of PBS) using a 27-gauge needle, which was positioned in the center of the tumor. The entire treatment volume was administered intratumorally over 15 s. The needle was then held in this position for 10 s and withdrawn slowly in order to prevent leakage of drug from the injection site. In vivo dosing was based on both literature review and our pilot studies that revealed antitumor activity (without clinical evidence of systemic toxicity) when a single dose of 0.25 mg of CPT was delivered by intratumoral injection.

### In Vivo Assessment of Combination CPT NP and CpG Therapy.

PDVC57 SCC tumors were induced with a subcutaneous injection of 5 million cells in 100 μl using a 27-gauge syringe. When tumors reached a diameter of 5 mm they were injected with either PBS (50 μL), blank BNPs (5 mg particles/50 μL) and CpG (10 μg), CPT suspended in intralipid (IL) (0.25 mg CPT/50 μL IL) and CpG (10 μg), NNP-CPT (0.25 mg CPT/50 μL) and CpG (10 μg), and BNP-CPT (0.25 mg CPT) and CpG (10 μg) using a 25-gauge needle positioned in the center of the tumor. Prior to injection, NPs were vortexed and sonicated using a Vibra Cell Sonicator for 30 seconds.

### Statistics.

The Mantel-Cox test was used to assess Kaplan-Meier curves (FIGS. 5 B and D). All other experimental groups were compared using an unpaired one-tailed Student t test with significance established at P<0.05. All statistical analyses were performed using GRAPHPAD PRISM™ 8.3 software.

### Results

To determine whether the improved intratumoral retention of BNP-CPT translated to decreased tumor burden in mice with SCCs, established tumors were treated with either blank BNPs, IL-CPT, NNP-CPT, or BNP-CPT (12.5 mg/kg CPT; FIG. 5A). Animals that received BNP-CPT treatment demonstrated significantly delayed tumor growth (FIG. 5B) and reduced tumor weight at harvest (FIG. 5C) when compared to control groups, confirming the advantages of BNPs in the treatment of this murine SCC model.

Tumor volumes at the time of treatment are evenly distributed among treatment groups.

Co-delivery of an adjuvant immunostimulatory agent, CpG, may enhance the capacity of BNP-CPT to eradicate tumors and extend animal survival by stimulating a local immune response to aid in tumor clearance, as has been well-characterized for CpG stimulation of TLR9 in other cutaneous malignancies, including melanoma (Najar, J. Invest. Dermatol. 128, 2204-2210 (2008); Koster et al., Clin. Cancer Res. 23, 5679-5686 (2017)); and cutaneous T cell lymphoma (CTCL) (Kim J. Am. Acad. Dermatol. 63, 975-983 (2010)). Through tumor induction and treatment, it was found that mice receiving combination therapy of BNP-CPT plus adjuvant CpG had significantly delayed tumor growth, and full, histologically confirmed resolution in 17% of the SCC tumors.

In a separate experiment, mice treated with BNP-CPT plus adjuvant CpG demonstrated significantly delayed tumor growth when compared to mice treated with vehicle, blank BNPs plus adjuvant CpG, or IL-CPT plus adjuvant CpG (FIG. 5D), and 20% of tumors resolved completely, confirming the therapeutic advantages of BNP delivery.

Mice treated with BNP-CPT plus CpG show enhanced survival when compared to BNP-CPT alone, with full resolution of a proportion of tumors. 6-7 mm tumors treated with BNP-CPT (12.5 mg/kg, 4.8% CPT loading) and adjuvant CpG (10 μg) demonstrated improved survival when compared to tumors treated with vehicle, or BNP-CPT alone. Resolution was assessed clinically, as well as histologically, and observed only within mice treated with combination therapy of BNP-CPT and CpG. PBS vs BNP-CPT+CpG*P<0.05, PBS vs BNP-CPT **P<0.01, Mantel-Cox test, n=7 (PBS), 8 (BNP-CPT), 6 (BNP-CPT+CpG).

These data show that local delivery of both chemotherapeutic and immunostimulatory agents represents a practical, nonsurgical approach to the treatment of KDCs.

This example shows that BNPs encapsulating CPT were very effective as a local, nonsurgical treatment for nodular squamous cell carcinoma (SCC) skin cancers in a mouse model. BNP-CPT treatment facilitated tumor destruction and resolution, and was compatible with local immunotherapy. These findings demonstrate that BNP delivery of antitumor agents may provide opportunities for nonsurgical treatment of nodular skin cancers like SCC. Results show that efficacy was more than additive when the BNP delivering chemotherapeutic agent were co-administered with immunostimulatory agents such as CPG.

The examples show that BNPs encapsulating CPT were very effective as a local, nonsurgical treatment for nodular squamous cell carcinoma (SCC) skin cancers in a mouse model. BNP-CPT treatment facilitated tumor destruction and resolution, and was compatible with local immunotherapy. These findings demonstrate that BNP delivery of antitumor agents can be effective for nonsurgical treatment of nodular skin cancers like SCC. Results show that efficacy was more than additive when the BNP delivering chemotherapeutic agent were co-administered with immunostimulatory agents such as CPG. Taken together, these results show that percutaneous delivery of a chemotherapeutic agent via BNPs, with or without adjuvant immunostimulation, represents a viable, nonsurgical alternative for treating cutaneous malignancy.

The data show the feasibility and augmented benefits of multimodal therapy in a single-dose treatment strategy for the local treatment of SCCs. Currently, the use of existing topical agents (e.g., 5-fluorouracil or imiquimod) for the treatment of superficial SCC requires significant patient compliance with multiple applications daily over a span of weeks to months. Furthermore, such topical agents do not provide for sufficient penetration or elimination of thicker, more nodular skin tumors. A single injection therapy, as in the case of BNP-CPT, can improve the logistics, safety, and efficacy of nonsurgical treatment of skin cancers like SCC.

Publications cited herein and the material for which they are cited are specifically incorporated by reference. Modifications and variations of the present invention will be obvious to those skilled in the art from the foregoing detailed description and are intended to be encompassed by the following claims.

1. An anti-tumor chemotherapeutic formulation comprising
bioadhesive nanoparticles (BNPs) with a polymer core and containing hyperbranched polyglycerol (HPG), having incorporated therein chemotherapeutic agent, and
one or more immunostimulant.

2. The formulation of claim 1 wherein the chemotherapeutic agent is incorporated into the core of the BNPs.

3. The formulation of claim 1 wherein the immunostimulant is attached to the surface of the BNPs.

4. The formulation of claim 1 wherein immunostimulant is not bound to the BNPs.

5. The formulation of claim 1 wherein the chemotherapeutic agent is selected from the group consisting of proteins, peptides, nucleic acid molecules, and organic molecules.

6. The formulation of claim 5 wherein the chemotherapeutic agent is selected from the group consisting of doxorubicin, cyclosporines, mitomycin C, cisplatins and carboplatins, Bis-chlorethylnitrosourea, carmustine, lomustine, semustine, 5-Fluorouracil, methotrexate, adriamycin, camptothecins, taxanes, mechlorethamine, cyclophosphamide, chlorambucil, dacarbazine, lomustine, carmustine, procarbazine, chlorambucil, ifosfamide, gemcitabine, cytosine arabinoside, fludarabine, and floxuridine, epothilones, vinca alkaloids, thalidomide, maytansines, auristatins, and combinations thereof.

7. The formulation of claim 5 wherein the chemotherapeutic agent is selected from the group consisting of antibodies inhibiting cell growth, proliferation or metastasis, receptor tyrosine kinase (RTK) inhibitors, and inhibitors of tyrosine kinase, transforming growth factor-α or transforming growth factor-β inhibitors.

8. The formulation of claim 5 wherein the chemotherapeutic agent is selected from the group of oligonucleotide drugs consisting of DNA, RNAs including mRNAs, antisense, siRNA, miRNA, anti-miRNA, piRNA, aptamers, ribozymes, external guide sequences for ribonuclease P, and triplex forming agents.

9. The formulation of claim 1 wherein the adjuvant is selected from the group consisting of TLR ligands such as CpG-containing oligonucleotides, lipopolysaccharide, single-stranded RNA, triphosphate-RNA, double-stranded RNA, imiquimod, resiquimod, polyinosinic:polycytidylic acid, flagellin, RIG-I-like receptors, checkpoint inhibitors such as those targeting PD-1, PDL-1, CTLA-4, and VISTA, immunostimulatory cytokines, bacterial or bacterial components or vaccines thereof, viral or viral components or vaccines thereof, fungi or fungi components or vaccines thereof, colony stimulating factors such as GM-CSF), type I and type II interferons such as interferon-alpha and interferon-gamma, interleukins such as interleukin-1 and interleukin-12, endogenous immunostimulants such as deoxycholic acid, defective interfering particles (DIPs) of viruses, and pattern recognition receptor (PRRs) agonists/ligands such as monophosphoryl lipid A and STING agonists.

10. The formulation of claim 4 wherein the BNPs are coadministered with an immunostimulatory agent.

11. The formulation of claim 10 where the chemotherapeutic agent and adjuvant are co-formulated.

12. The formulation of claim 10 where the chemotherapeutic agent is encapsulated in the BNP and the immunostimulatory agent is co-administered in excipient with BNP, separate from BNP.

13. The formulation of claim 1 in a microneedle array.

14. A method of treating a patient with cancers that form collections of cells that may be targeted with local delivery, especially primary tumors of the skin or metastatic tumors to the skin, and tumor or metastatic collection of cancer cells that may be accessed within any other organ or tissue comprising administering the formulation of claim 1.

15. The method of claim 14 wherein the formulation is administered via microneedle delivery, needle injection, trochanter delivery, intravascular deliver, or via surgical accessibility.

16. The method of claim 14 wherein the formulation is administered directly to the tumor or adjacent to the tumor in the skin, including intratumorally, intradermally, and subcutaneously; and to any other tumor and site accessible with intratumoral injection or the area surrounding the tumors, including via infusion into vascular element that perfuse the tumor.

17. The method of claim 14 wherein the formulation is administered to an individual with a cutaneous cancer.

18. The method of claim 17 wherein the cancer is selected from the group consisting of basal cell carcinoma, squamous cell carcinoma, melanoma, Primary cutaneous lymphoma, Merkel cell carcinoma, Kaposi sarcoma, adnexal tumor, and other metastatic cancers to the skin.

19. The method of claim 14 wherein the formulation is administered more than once and/or in multiple locations.

20. The method of claim 14 wherein the formulation is administered to an immunocompetent person in need thereof.
